(19) United States
(12) Patent Application Publication
(10) Pub. No.: US 2017/0087096 A1
(43) Pub. Date: Mar. 30, 2017

(54) NANOCAPSULAR FORMULATION OF ACTIVE PHARMACEUTICAL INGREDIENTS

(71) Applicants: SANOFI, Paris (FR); UNIVERSIDADE DE SANTIAGO DE COMPOSTELA, Santiago de Compostela (ES)

(72) Inventors: Gudrun Lenherr, Frankfurt am Main (DE); Noemi Csaba, Santiago de Compostela (ES); Nomceba Lungile Thwala, Santiago de Compostela (ES); Zhigao Niu, Santiago de Compostela (ES); Maria José Alonso, Santiago de Compostela (ES)

(21) Appl. No.: 15/311,818

(22) PCT Filed: Jun. 12, 2015
(86) PCT No.: PCT/EP2015/063164

§ 371 Date: Nov. 16, 2016

(30) Foreign Application Priority Data
Jun. 13, 2014 (EP) 14305899.8

(51) Int. Cl. A61K 9/51; A61K 38/28

(52) U.S. Cl. CPC A61K 9/5169; A61K 38/28; A61K 9/5146; A61K 9/5123

(57) ABSTRACT

Nanocapsule systems of at least one active pharmaceutical ingredient selected from the group consisting of at least one insulin, insulin analogue, insulin derivative, glucagon-like peptide-1 receptor agonist (GLP1 R agonist) and/or dual GLP-1 receptor/glucagon receptor agonist and/or or any combination thereof are disclosed.

The present invention relates to a nanocapsule system, a pharmaceutical composition that comprises said nanocapsule system and a kit comprising said nanocapsule system. It also relates to said nanocapsule system for use in the treatment and related method of treatments. The present invention also relates to methods for producing said nanocapsules.

### BACKGROUND OF THE INVENTION

Diabetes mellitus is a metabolic disorder in which the ability to utilize glucose is more or less completely lost. For decades, insulin has been used in the treatment of diabetes mellitus. Several insulin formulations have been developed, e.g. insulin zinc (Zn (II)) suspension, formulations containing protamine, etc. Further, the active pharmaceutical ingredient insulin itself has been modified by developing fast acting insulin analogues (e.g. insulin aspart, insulin lispro, insulin glulisine) and long acting insulin analogues and derivatives (e.g. insulin detemir, insulin degludec, insulin glargin). Fast acting insulin preparations are usually solutions of insulin, while long acting insulin preparations can be suspensions containing insulin in crystalline and/or amorphous form precipitated by the addition of zinc (Zn(II)) salts (e.g. zinc chloride) alone or by addition of protamine or by a combination of both.

Insulins are administered parenterally. Non-invasive administration routes like oral delivery of insulin are investigated but there are several hurdles, as such enzymatic degradation in the gastrointestinal tract, drug efflux pumps, insufficient and variable absorption from the intestinal mucosa, as well as first pass metabolism in the liver.

US2013/058999 (WO 2011/086093) discloses liquid pharmaceutical compositions comprising at least one insulin peptide, at least one semi-polar protic organic solvent and at least two non-ionic surfactants.

US2012/0121670 (WO 2010/7122204) discloses a system for administering active ingredients comprising nanocapsules with a diameter less than 1 μm comprising a polyarginine salt, a negative phospholipid and an oil.

US 2014/0023703 (WO 2012/095543) discloses a system for the administration of active ingredients comprising nanocapsules which comprise an oil, a cationic surfactant and a specific polymer.

PCT/ES2013/070885 discloses nanocapsule systems suitable for administering active ingredients.

Shukla et al.; Expert Opinion Drug Delivery, 2010, Vol. 7(09), pp. 993-1011 relates to polyelectrolyte-based surrogate carriers for delivery of bioactives.

The incorporation of active pharmaceutical ingredients in systems of nanometric size leads to improvements in solubility, protection from degradation or greater penetration of the active pharmaceutical ingredients either by nanoencapsulation of active molecules or adsorption thereof on the polymer coating. Moreover, it is also known that the capacity of these systems for crossing external barriers and reaching the interior of the body depends both on their size and on their composition. Particles of small size will increase the degree of transport relative to those of a larger size.

It has now surprisingly been found that alternative nanocapsule systems comprising a surface layer that comprises a cationic charged polymer, optionally a second surface layer that comprises a negatively charged polymer, a lipid core that comprises at least one lipophilic compound, a suitable surfactant and at least one active pharmaceutical ingredient selected from the group consisting of insulin, insulin analogues, insulin derivatives, glucagon-like peptide-1 receptor agonist (GLP1R agonist) and dual GLP-1 receptor/glucagon receptor agonist and any combination thereof, show improved properties. This nanocapsule system is stable, easy to obtain and moreover allows effective combination of active pharmaceutical ingredients of different kinds, both hydrophilic and lipophilic, in particular insulin, insulin analogues and/or insulin derivatives, glucagon-like peptide-1 receptor agonist (GLP1 R agonist) and/or dual GLP-1 receptor/glucagon receptor agonist and/or or any combination thereof.

The nanocapsule system of the present invention provides improved properties in comparison with other systems for administration and/or release of drugs, owing to its particular behaviour with respect to:
encapsulation/combination of active pharmaceutical ingredients: the system can include one or more active pharmaceutical ingredients or adjuvants, hydrophilic or lipophilic, in proportions greater than that of nanoparticles, micelles, complexes, nanogels;
release of the active pharmaceutical ingredient: not only the lipophilic core, but also the polymer coating layer has an effect on its rate of release, allowing controlled release of active principle according to the application and needs;
stability in biological fluids: the polymer coating endows the lipid core with great stability, which represents an advantage relative to other systems of micro- and nanoemulsions;
specific interaction with particular biological surfaces: the polymer coating endows the lipid core with the possibility of interacting with mucosal surfaces as well as with epithelia and specific cells;
the rapid metabolization and excretion of e.g. protamine as cationic polymer gives this system a pharmacokinetic safety profile, and the same profile cannot be demonstrated for other nanocapsule systems with some other type of coating;
stability during lyophilisation.

### SUMMARY OF THE INVENTION

One embodiment of the present invention relates to a nanocapsule system that comprises:
a. a surface layer that comprises a cationic charged polymer;
b. optionally a second surface layer that comprises a negatively charged polymer;
b. a lipid core that comprises at least one lipophilic compound;
c. a surfactant, a mixture of surfactants or at least one surfactant; and
d. at least one active pharmaceutical ingredient selected from the group consisting of insulin, insulin analogue, insulin derivative, glucagon-like peptide-1 receptor agonist (GLP1R agonist), dual GLP-1 receptor/glucagon receptor agonist and any combination thereof.

In another embodiment, the nanocapsule system according to the present invention comprises a surface layer that comprises a cationic charged polymer which is selected from the group consisting of polyaminoacids, polypeptides, polylysine, polyarginine, lixisenatide,protamine and combinations thereof.

In another embodiment, the nanocapsule system according to the present invention comprises a second surface layer that comprises a negatively charged polymer which is selected from the group of polyacids and derivatives, polysialic acid, polyacrylic acid, hyaluronic acid, polyglutamic acid, alginic acid, polyglucuronic acid, and xanthan gum.

In another embodiment, the nanocapsule system according to the present invention comprises at least one surfactant or a mixture of surfactants which is characterized by possessing a hydrophilic-lipophilic balance (HLB) above 8, in another embodiment above 9, above 10, above 11, above 12, above 13, or 14.

In another embodiment, the nanocapsule system according to the present invention comprises at least one surfactant selected from the group consisting of sorbitan esterified with at least one fatty acid and ethoxylates thereof, fatty acid esters, fatty acid salts, gum tragacanth, bile salts and bile salt derivatives and Poloxamers.

In another embodiment, the nanocapsule system according to the present invention comprises a surfactant, a mixture of surfactants or at least one surfactant which is an sorbitan esterified with at least one fatty acid and ethoxylates thereof selected from the group consisting of sorbitan monooleate (Span® 80, HLB 4.3), sorbitan monolaurate (Span® 20, HLB 8.6), sorbitan monostearate (Span® 60, HLB 4.7), sorbitan trioleate (Span® 85, HLB 1.8), sorbitan sesquiolate (Span® 83, HLB 3.7), sorbitan monopalmitate (Span® 40, HLB 6.7), sorbitan isostearate (Span® 120, HLB 4.7), polyoxyethylene sorbitan monooleate (polysorbate 80; Tween° 80; HLB 15), polyoxyethylene sorbitan monolaurate (polysorbate 20; Tween® 20; HLB 16.7 and Tween 21®; HLB 13.3), polyoxyethylene sorbitan monopalmitate (Tween® 40, HLB 15.6), polyoxyethylene sorbitan monostearate (Tween® 60, HLB 14.9 and Tween 61®; HLB 9.6), polyoxyethylene sorbitan monooleate (Tween 81®; HLB 10), polyoxyethylene sorbitan tristearate (Tween 65®; HLB 10.5), polyoxyethylene sorbitan trioleate (Tween 85®; HLB 11), polyoxyethylene sorbitan monolaurate (Tween® 20, HLB 16.7 and Tween 21®; HLB 13.3) or any combination thereof.

In another embodiment, the nanocapsule system according to the present invention comprises a surfactant, a mixture of surfactants or at least one surfactant which is a fatty acid ester or a fatty acid salt selected from the group consisting of PEGylated fatty acid esters and mixtures with PEG, polyethylene glycol monostearate (HLB 11.6), polyethylene glycol stearate, polyethylene glycol stearate 40 (HLB 17), polyethylene glycol dilaurate 400 (HLB 9.7), polyethylene glycol dilaurate 200 (HLB 5.9), polyethylene glycol monopalmitate (HLB 11.6), polyethylene glycol stearate, polyethylene glycol stearate 40 (HLB 16.9) polyethylene glycol stearate 100 (HLB 18.8), Solutol H515® (HLB 15), polyethylene glycol-15-hydroxystearate (HLB 14-16), D-alpha-tocopheryl polyethylene glycol succinate (TPGS; HLB 13.2), triethanolammonium oleate (HLB 12), sodium oleate (HLB 18), sodium lauryl sulphate (HLB 40), triethanolamine oleate (HLB 12) and lithium dodecyl sulfate (HLB 16-18), sodium oleate (HLB 18), sodium dodecyl sulphate (HLB 40), sodium lauryl sulphate (HLB 40) or any combination thereof.

In another embodiment, the nanocapsule system according to the present invention comprises at least one surfactant which is gum tragacanth (HLB 11.9).

In another embodiment, the nanocapsule system according to the present invention comprises at least one surfactant a mixture of surfactants or at least one surfactant, which is a bile salt and/or bile salt derivative selected from the group consisting of sodium cholate (HLB 18), sodium deoxycholate (HLB 16), sodium glycocholate (HLB 16-18), sodium taurocholate (HLB 16), sodium taurodeoxycholate (HLB 20.1).

In another embodiment, the nanocapsule system according to the present invention comprises a surfactant, a mixture of surfactants or at least one surfactant which is a poloxamer selected from the group consisting of Poloxamer 124 (HLB 16), Poloxamer 188 (HLB 29), Poloxamer 237 (HLB 29), Poloxamer 238 (HLB 28), Poloxamer 278 (HLB 28), Poloxamer 338 (HLB 27), and Poloxamer 407 (HLB 22).

In another embodiment, the nanocapsule system according to the present invention comprises a lipid core that comprises at least one lipophilic compound which is selected from the group consisting of fatty acids and their esters with glycerides, and terpenoids.

In another embodiment, the nanocapsule system according to the present invention comprises a lipid core that comprises at least one lipophilic compound which is selected from the group consisting of peanut oil, cottonseed oil, olive oil, castor oil, soybean oil, safflower oil, geranium oil, palm oil, alpha-tocopherol (vitamin E), oleic acid, linoleic acid, isopropyl myristate, squalene, Miglyol®, Labrafil®, Labrafac®, Peceol®, Maisine®, caprylic/capric triglyceride, linoleoyl macrogol-6 glycerides (corn oil PEG-6 esters), triglycerides medium chain, glyceryl oleate, glyceryl linoleate, glycerol monooleate and any combination thereof. Miglyol® (CAS-NO 52622-27-2) is the brand name of a commercial product containing a mixture of decanoyl- and octanoyl glycerides. The INCI name is “Caprylic/Capric Triglyceride”. Labrafil® is the brand name of a commercial product containing oleoyl macrogol-6 glycerides. Labrafac® is the brand name of a commercial product containing medium chain fatty acid triglycerides; synonym is ‘Caprylic/Capric triglyceride’ (HLB 1). Peceol® is the brand name of a commercial product containing glycerol monooleates (type 40). Maisine® is the brand name of a commercial product containing glycerol monolinoleate.

In another embodiment, the lipophilic compound is selected from the group consisting of squalene oil, flavoring oils, silicone oil, essential oils, water-insoluble vitamins, isopropyl stearate, butyl stearate, octyl palmitate, cetyl palmitate, tridecyl behenate, diisopropyl adipate, dioctyl sebacate, menthyl anthranilate, cetyl octanoate, octyl salicylate, isopropyl myristate, ketols of neopentyl glycol dicaprate, Cerafilos®, decyl oleate, C₁₂-C₁₅ alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, liquid paraffins, isododecane, petroleum jelly, argan oil, colza oil, chili oil, coconut oil, corn oil, cottonseed oil, linseed oil, grapeseed oil, mustard oil, olive oil, palm oil, fractionated palm oil, peanut oil, castor oil, pine seed oil, poppy seed oil, pumpkin seed oil, rice bran oil, safflower oil, tea oil, truffle oil, vegetable oil, apricot oil, jojoba oil, macadamia oil, wheatgerm oil, almond oil, soybean oil, sesame oil, hazelnut oil, sunflower oil, hemp oil, bois oil, kukui nut oil, avocado oil, walnut oil, fish oil, berry oil, Jamaican pepper oil, juniper oil, seed oil, almond seed oil, aniseed oil, celery seed oil, cumin seed oil, nutmeg seed oil, basil leaf oil, bay leaf oil, cinnamon leaf oil, common sage leaf oil, eucalyptus leaf oil, lemon leaf oil, tea tree oil, oregano oil, patchouli leaf oil, mint leaf oil, pine needle oil, rosemary leaf oil, spearmint oil, tea tree leaf oil, thyme leaf oil, Canada tea leaf oil, blossom oil, chamomile oil, Roman sage oil, clove oil, geranium blossom oil, hyssop blossom oil, jasmine blossom oil, lavender blossom oil, mauka blossom oil, marjoram blossom oil, orange blossom oil, rose blossom oil, ylang-ylang blossom oil, bark oil, cassia bark oil, cinnamon bark oil, sassafras bark oil, wood oil, camphor wood oil, cedar wood oil, rose stalk oil, sandalwood oil, ginger wood oil, resin oil, castor oil, myrrh oil, peel oil, Bergamo peel oil, grapefruit peel oil, lemon peel oil, lime peel oil, orange peel oil, mandarin peel oil, root oil, valerian oil, oleic acid, linoleic acid, oleyl alcohol, isostearyl alcohol, ethyl oleate, Miglyol®, Labrafil®, Labrafac®, Rylo®, Peceol® and Maisine®, synthetic or semisynthetic derivatives thereof and combinations thereof. Preferably the oil is selected from the group consisting of peanut oil, cottonseed oil, olive oil, castor oil, soybean oil, safflower oil, palm oil, α-tocopherol (vitamin E), isopropyl myristate, squalene, Miglyol®, Labrafil®, Labrafac®, Peceol® and Maisine® or mixtures thereof. More preferably, the oils are Miglyol®, squalene or α-tocopherol.

In another embodiment, the nanocapsule system according to the present invention comprises a lipid core that comprises at least one lipophilic compound, wherein the lipophilic compound is selected from the group consisting of oleic acid, caprylic/capric triglyceride, squalene or alpha-tocopherol.

In another embodiment, the nanocapsule system according to the present invention comprises an active pharmaceutical ingredient which is human insulin.

In another embodiment, the nanocapsule system according to the present invention comprises an active pharmaceutical ingredient which is an insulin analogue selected from the group consisting of insulin aspart, insulin lispro, insulin glargine and insulin glulisine or any combinations thereof.

In another embodiment, the nanocapsule system according to the present invention comprises an active pharmaceutical ingredient which is an insulin derivative selected from the group consisting of insulin detemir and insulin degludec.

In another embodiment, the nanocapsule system according to the present invention comprises an active pharmaceutical ingredient which is a human insulin stabilized towards proteolytic degradation. In another embodiment, the nanocapsule system according to the present invention comprises an active pharmaceutical ingredient which is human insulin stabilized towards proteolytic degradation wherein said human insulin stabilized towards proteolytic degradation contains at least more than one amino acid substitution in the A and B chain and/or at least one deletion or extension of the C- and N-termini and/or at least one functionalized N-termini (e.g. di alkylation) and/or at least one acylation at K(epsilon)-NH₂ and/or at least one introduction of at least one additional disulfide bond. Suitable human insulins stabilized towards proteolytic degradation are disclosed in the following document which are incorporated by reference: WO2008/034881, WO2008/145721, WO2009/112583, WO2009/115469, WO2012/049307, and WO2013/093009. In one embodiment, such human insulin stabilized towards proteolytic degradation is A14E, B25H, B29K(N-epsilon Octodecanedioyl-gamma Glutamic acid-OEG-OEG), desB30 human insulin as disclosed in WO 2009/115469; the term “OEG” is a short notation for the amino acid NH₂(CH₂)₂O (CH₂)₂OCH₂CO₂H.

In another embodiment, the nanocapsule system according to the present invention comprises an active pharmaceutical ingredient which is a glucagon-like peptide-1 receptor agonist (GLP1R agonist) selected from the group consisting of exendin-4, liraglutide, lixisenatide, dulaglutide, albiglutide, semaglutide, and taspoglutide or any combinations thereof.

In another embodiment, the nanocapsule system according to the present invention comprises an active pharmaceutical ingredient which is a dual GLP-1 receptor/glucagon receptor agonist.

In another embodiment, the present invention relates to a pharmaceutical composition that comprises
a. a surface layer that comprises a cationic charged polymer;
b. optionally a second surface layer that comprises a negatively charged polymer;
c. a lipid core that comprises at least one lipophilic compound;
d. a surfactant or at least one surfactant or a mixture of surfactants; and
e. at least one active pharmaceutical ingredient selected from the group consisting of at least one insulin, insulin analogue, insulin derivative, glucagon-like peptide-1 receptor agonist (GLP1R agonist) and/or dual GLP-1 receptor/glucagon receptor agonist and/or or any combination thereof; and
f. optionally one or more pharmaceutically acceptable excipients.

In another embodiment, the present invention relates to a pharmaceutical composition that comprises
a. a surface layer that comprises a cationic charged polymer;
b. optionally a second surface layer that comprises a negatively charged polymer;
c. a lipid core that comprises at least one lipophilic compound;
d. a surfactant or at least one surfactant or a mixture of surfactants characterized by possessing a hydrophilic-lipophilic balance (HLB) above 8, above 9, above 10, above 11, above 12, above 13, or 14; and
e. at least one active pharmaceutical ingredient selected from the group consisting of at least one insulin, insulin analogue, insulin derivative, glucagon-like peptide-1 receptor agonist (GLP1R agonist) and/or dual GLP-1 receptor/glucagon receptor agonist and/or or any combination thereof; and
f. optionally one or more pharmaceutically acceptable excipients.

In another embodiment, the pharmaceutical composition according to the present invention comprises one or more further active pharmaceutical ingredients.

In another embodiment, the nanocapsule system according to the present invention or the pharmaceutical composition according to the present invention is lyophilized.

In another embodiment, the present invention relates to a kit comprising one or more separate packages of the nanocapsule system according to the present invention or the pharmaceutical composition according to the present invention and at least one further active pharmaceutical ingredient and/or a medical device.

In another embodiment, the nanocapsule system according to the present invention and/or the pharmaceutical composition according the present invention and/or the kit according to the present invention is for use in the treatment of diabetes mellitus.

In another embodiment, the nanocapsule system according to the present invention and/or the pharmaceutical composition according the present invention and/or the kit according to the present invention is for use in the treatment of hyperglycemia.

In another embodiment, the nanocapsule system according to the present invention and/or the pharmaceutical composition according the present invention and/or the kit according to the present invention is for use in lowering blood glucose level.

In another embodiment, the nanocapsule system according to the present invention and/or the pharmaceutical composition according the present invention and/or the kit according to the present invention is for subcutaneous, transdermal, buccal, oral, pulmonary or nasal administration.

In another embodiment, the present invention relates to a method of treating diabetes mellitus in a subject in need thereof comprising administering the nanocapsule system according to the present invention or the pharmaceutical composition according to the present invention.

In another embodiment, the present invention relates to a method of treating hyperglycemia in a subject in need thereof comprising administering the nanocapsule system according to the present invention or the pharmaceutical composition according to the present invention.

In another embodiment, the present invention relates to a method of lowering blood glucose levels in a subject in need thereof comprising administering the nanocapsule system according to the present invention or the pharmaceutical composition according to the present invention.

In another embodiment, the present invention relates to a medical device for administering the nanocapsule system according to the present invention or the pharmaceutical composition according to the present invention.

In another embodiment the present invention relates to a method for producing the nanocapsule system according to the present invention comprising the emulsification of an internal lipophilic phase containing the lipophilic compounds, and an external water phase containing a cationic charged polymer, and optionally the surfactant or the mixture of surfactants. At least one active pharmaceutical ingredient selected from the group consisting of human insulin, insulin analogue, insulin derivative, glucagon-like peptide-1 receptor agonist (GLP1R agonist), dual GLP-1 receptor/glucagon receptor agonist, and any combination thereof, is incorporated into the internal lipophilic phase, into the external aqueous phase, or both. Optionally, a negatively charged polymer is added into the external aqueous phase upon deposition of the cationic polymer layer or after adsorption of the active ingredient onto the cationic polymer layer.

In another embodiment the present invention relates to a method for producing the nanocapsule system according to the present invention comprising the emulsification of an internal lipophilic phase containing the lipophilic compounds, and an external water phase containing a cationic charged polymer, and optionally the surfactant or the mixture of surfactants. At least one active pharmaceutical ingredient selected from the group consisting of human insulin, insulin analogue, insulin derivative, glucagon-like peptide-1 receptor agonist (GLP1R agonist), dual GLP-1 receptor/glucagon receptor agonist, and any combination thereof, is incorporated into the internal lipophilic phase. Wherein, the active pharmaceutical ingredient is dispersed in the lipophilic phase either as a powder or as an aqueous solution.

In another embodiment the present invention relates to a method for producing the nanocapsule system according to the present invention comprising the emulsification of an internal lipophilic phase containing the lipophilic compounds, and an external water phase containing a cationic charged polymer, and optionally the surfactant or the mixture of surfactants. At least one active pharmaceutical ingredient selected from the group consisting of human insulin, insulin analogue, insulin derivative, glucagon-like peptide-1 receptor agonist (GLP1R agonist), dual GLP-1 receptor/glucagon receptor agonist, and any combination thereof, is incorporated into the internal lipophilic phase. Wherein, the at least one active pharmaceutical ingredient is complexed with the surfactant or any of the surfactants in a mixture of surfactants.

In another embodiment, the present invention relates to a method for producing the nanocapsule system according to the present invention comprising the emulsification of an internal lipophilic phase containing the lipophilic compound and an external water phase containing the cationic charged polymer, optionally a negatively charged polymer, the surfactant or the mixture of surfactants, and the at least one active pharmaceutical ingredient selected from the group consisting of human insulin, insulin analogue, insulin derivative, glucagon-like peptide-1 receptor agonist (GLP1R agonist), dual GLP-1 receptor/glucagon receptor agonist, and any combination thereof, wherein the at least one active pharmaceutical ingredient is dispersed either in the lipophilic phase either as a powder or as an aqueous solution, wherein the at least one active pharmaceutical ingredient is complexed with the surfactant or any of the surfactants in a mixture of surfactants.

In another embodiment, the present invention relates to a method for producing the nanocapsule system according to the present invention comprising the following steps (single-step solvent diffusion method):
a) preparing an aqueous solution that comprises the cationic polymer, in one embodiment protamine, and optionally a water-soluble surfactant;
b) preparing a solution, in one embodiment a solution, that comprises an oil and one or more surfactants, characterized by possessing a hydrophilic-lipophilic balance above 8, above 9, above 10, above 11, above 12, above 13, or 14;
c) mixing the solutions prepared in steps a) and b), with stirring, the nanocapsules being obtained spontaneously; and
d) optionally, completely or partially evaporating the organic solvents from the mixture obtained in the preceding step.

In another embodiment, the present invention relates to a method for producing the nanocapsule system according to the present invention comprising the following steps (single-step solvent diffusion method):
a) preparing an aqueous solution that comprises the cationic polymer, in one embodiment protamine, and optionally a water-soluble surfactant;
b) preparing a solution, in one embodiment an organic solution, that comprises an oil and one or more surfactants, characterized by possessing a hydrophilic-lipophilic balance above 8, above 9, above 10, above 11, above 12, above 13, or 14;
c) mixing the solutions prepared in steps a) and b), with stirring, the nanocapsules being obtained spontaneously; and
d) optionally, completely or partially evaporating the organic solvents from the mixture obtained in the preceding step; and
e) adding a negatively charged polymer in order to from a second layer onto the cationic layer of the nanocapsules.

In another embodiment, the present invention relates to a method for producing the nanocapsule system according to the present invention (two-step solvent diffusion method) that comprises coating a nanoemulsion with protamine by an incubation process with an aqueous solution of the polymer.

In another embodiment, the present invention relates to a method for producing the nanocapsule system according to the present invention wherein the incubation process comprises mixing the nanoemulsion with an aqueous solution of the coating polymer. Said nanoemulsion consists of at least one oil, one or more surfactants, and an aqueous phase. The aqueous phase can contain other surfactants, salts, and other auxiliaries.

In another embodiment, the present invention relates to a method for producing the nanocapsule system according to the present invention wherein the incubation process comprises mixing the nanoemulsion with an aqueous solution of the coating polymer. Said nanoemulsion consists of at least one oil, one or more surfactants characterized by possessing a hydrophilic-lipophilic balance above 8, above 9, above 10, above 11, above 12, above 13, or 14, and an aqueous phase. The aqueous phase can contain other surfactants, salts, and other auxiliaries.

In another embodiment, the present invention relates to a method for producing the nanocapsule system according to the present invention comprising the following steps (solvent diffusion method):
a) preparing a solution, in one embodiment a solution, that comprises an oil, and one or more surfactants; and
b) adding the solution obtained in step a) to an aqueous phase that optionally contains a water-soluble surfactant and is kept stirred to form a nanoemulsion; and
c) optionally, completely or partially evaporating the in one embodiment present organic solvents; and
d) adding a cationic polymer to the nanoemulsion resulting in step b).

In another embodiment, the present invention relates to a method for producing the nanocapsule system according to the present invention comprising the following steps (solvent diffusion method):
a) preparing a solution, in embodiment an solution, that comprises an oil, and one or more surfactants characterized by possessing a hydrophilic-lipophilic balance above 8, above 9, above 10, above 11, above 12, above 13, or 14; and
b) adding the solution obtained in step a) to an aqueous phase that optionally contains a water-soluble surfactant and is kept stirred to form a nanoemulsion; and
c) optionally, completely or partially evaporating the in one embodiment present organic solvents; and
d) adding a cationic polymer to the nanoemulsion resulting in step b).

In another embodiment, the present invention relates to a method for producing the nanocapsule system according to the present invention comprising the following steps (sonication method):
a) preparing a solution, in one embodiment a solution, that comprises an oil, and one or more surfactants characterized in that they possess a hydrophilic-lipophilic balance above 8; and
b) adding the solution obtained in step a) to an aqueous phase that optionally contains a water-soluble surfactant and sonicating; and
c) optionally, completely or partially evaporating the in one embodiment present organic solvents; and
d) adding a cationic polymer to the nanoemulsion resulting in step b).

In another embodiment, the present invention relates to a method for producing the nanocapsule system according to the present invention comprising the following steps (homogenization method):
a) preparing a solution, in one embodiment an organic solution, that comprises an oil, and one or more surfactants;
b) adding the solution obtained in step a) to an aqueous phase that optionally contains a water-soluble surfactant and homogenizing;
c) diluting the emulsion obtained in step b) with water and homogenizing;
d) optionally, completely or partially evaporating the in one embodiment present organic solvents;
e) adding a cationic polymer to the nanoemulsion resulting in step c).

In another embodiment, the present invention relates to a method for producing the nanocapsule system according to the present invention comprising the following steps (homogenization method):
a) preparing a solution that comprises an oil, and one or more surfactants characterized by possessing a hydrophilic-lipophilic balance above 8, above 9, above 10, above 11, above 12, above 13, or 14; and
b) adding the solution obtained in step a) to an aqueous phase that optionally contains a water-soluble surfactant and homogenizing; and
c) diluting the emulsion obtained in step b) with water and homogenizing; and
d) optionally, completely or partially evaporating the organic solvents to constant volume; and
e) adding a cationic polymer to the nanoemulsion resulting in step c).

In another embodiment, the present invention relates to a method for producing the nanocapsule system according to the present invention, wherein an organic solvent is introduced in the lipid solution, this organic solvent being in one embodiment a mixture of polar solvents such as ethanol, isopropanol and acetone and can further include non-polar solvents such as for example dichloromethane. The oil and the surfactant or surfactants, characterized by possessing a hydrophilic-lipophilic balance above 8, are incorporated in this organic phase. In one embodiment, the active pharmaceutical ingredient is also incorporated.

In another embodiment, the present invention relates to a method for producing the nanocapsule system according to the present invention, wherein the protamine is selected as cationic polymer, comprising the following steps:
a) preparing an aqueous solution of 20 ml at 0.05% w/v of protamine; and
b) preparing a lipophilic phase consisting of ethanol/acetone solution of one or both surfactants (sodium cholate and/or PEG stearate), to which Miglyol® 812 is added; and
c) mixing the solutions resulting from steps a) and b), with stirring, the nanocapsules being obtained spontaneously; and
d) optionally, evaporating the organic solvents from the mixture obtained in the preceding step.

In another embodiment, the present invention relates to a method for producing the nanocapsule system according to the present invention, wherein polyarginine is selected as cationic polymer, comprising the following steps:
a) preparing an aqueous solution of 20 ml containing 0.05% w/v of polyarginine and 0.25% (w/v) of poloxamer 188; and
b) preparing a lipophilic phase consisting of the ethanol/acetone solution of one or both surfactants (sorbitan monooleate and/or sodium deoxycholate), to which oleic acid is added; and
c) mixing the solutions resulting from steps a) and b), with stirring, the nanocapsules being obtained spontaneously; and
d) optionally, evaporating the organic solvents from the mixture obtained in the preceding step.

In another embodiment, the present invention relates to a method for producing the nanocapsule system according to the present invention comprising the following steps:
a) preparing a solution, in one embodiment an solution, that comprises an oil, and one or more surfactants; and
b) adding the solution obtained in step a) to an aqueous phase that optionally contains a water-soluble surfactant and is kept stirred to form a nanoemulsion; and
c) optionally, completely or partially evaporating the in one embodiment present organic solvents.

In another embodiment, the present invention relates to a method for producing the nanocapsule system according to the present invention, comprising an additional step of lyophilization, for the purpose of preserving them during storage so that they keep their initial characteristics. Owing to the nature of the coating of the nanocapsules of the present invention, as well as their characteristics, it is not necessary to use cryoprotectants during lyophilization.

Another additional advantage is that it is not necessary to dilute the colloidal system before lyophilization, since the nanocapsule systems of the invention do not form aggregates during reconstitution of the lyophilizate. Alternatively, it is possible to add one or more sugars that exert a cryoprotectant effect (examples of cryoprotectant agents included, but are not limited to, the following: trehalose, glucose, sucrose, mannitol, maltose, polyvinyl pyrrolidone (PVP). In the lyophilized form, the nanocapsules can be stored for long periods of time, and can easily be regenerated, if necessary, simply by adding an optimum volume of water.

### DETAILED DESCRIPTION

As used herein, the singular forms “a”, “an”, and “the” include plural reference unless the context clearly dictates otherwise. Thus, for example, reference to a fill material containing “a carrier” includes one or more carriers, reference to “an additive” includes reference to one or more of such additives.

As used herein, the term “active pharmaceutical ingredient” (API) includes any pharmaceutically active chemical or biological compound and any pharmaceutically acceptable salt thereof and any mixture thereof, that provides some pharmacologic effect and is used for treating or preventing a condition. Exemplary pharmaceutically acceptable salts include, but are not limited to, hydrochloric, sulfuric, nitric, phosphoric, hydrobromic, maleric, malic, ascorbic, citric, tartaric, pamoic, lauric, stearic, palmitic, oleic, myristic, lauryl sulfuric, naphthalinesulfonic, linoleic, linolenic acid, and the like. As used herein, the terms “active pharmaceutical ingredient”, “drug”, “active agent”, “active ingredient”, “active substance” and “drug” are meant to be synonyms, i.e., have identical meaning. In one embodiment of the present invention the active pharmaceutical ingredient is an antidiabetic agent. Examples of antidiabetic agents include bit are not limited to the following: insulins, insulin analogues, insulin derivatives, glucagon-like peptide-1 receptor agonist (GLP1R agonist), GLP-1 analogues, and GLP-1 receptor agonists and/or dual GLP-1 receptor/glucagon receptor agonist and/or or any combination thereof, as described in detail in the following:
a) insulin, insulin analogues, and insulin derivatives
   Examples of insulin, insulin analogues, and insulin derivatives include but are not limited to short-acting insulins; long-acting insulins; insulin glargine (Lantus®), insulin glulisine (Apidra®), insulin detemir (Levemir®), insulin lispro (Humalog®/Liprolog®), insulin degludec (Tresiba®), insulin aspart (NovoLog®/NovoRapid®), basal insulin and analogues (e.g. LY2605541, LY2963016), PEGylated insulin lispro, human insulin (Insuman®);
b) glucagon-like peptide-1 receptor agonist (GLP1R agonist), GLP-1 analogues, and GLP-1 receptor agonists
   Examples of GLP-1, GLP-1 analogues and GLP-1 receptor agonists include but are not limited to lixisenatide (AVE0010/ZP10/Lyxumia®), exenatide/exendin-4 (Byetta®/Bydureon®/ITCA 650), liraglutide (Victoza®), semaglutide, taspoglutide, albiglutide, dulaglutide, exenatide-XTEN and glucagon-XTEN, and polymer bound GLP-1 and GLP-1 analogues.

The proportion of active pharmaceutical ingredient incorporated in the nanocapsule systems according to the present invention can be up to approximately 50 wt % relative to the total dry weight of the components of the system of nanocapsules. However, the appropriate proportion will depend in each case on the active pharmaceutical ingredient that is to be incorporated, the indication for which it is used and the efficiency of administration. In a particular embodiment, the proportion of active principle can be up to approximately 10 wt %, preferably up to approximately 5 wt %. In one embodiment, the nanocapsule systems according to the present invention comprises more than one active pharmaceutical ingredient, which can be dissolved in the same solution or separately, depending on the nature of the active pharmaceutical ingredient to be incorporated.

As used herein, the terms “analogue of insulin” and “insulin analogue” refer to a polypeptide which has a molecular structure which formally can be derived from the structure of a naturally occurring insulin, for example that of human insulin, by deleting and/or exchanging at least one amino acid residue occurring in the naturally occurring insulin and/or adding at least one amino acid residue. The added and/or exchanged amino acid residue can either be codeable amino acid residues or other naturally occurring residues or purely synthetic amino acid residues. Examples of analogues of insulin include, but are not limited to, the following:

(i). ‘Insulin aspart’ is created through recombinant DNA technology so that the amino acid B28 in human insulin (i.e. the amino acid no. 28 in the B chain of human insulin), which is proline, is replaced by aspartic acid;

(ii). ‘Insulin lispro’ is created through recombinant DNA technology so that the penultimate lysine and proline residues on the C-terminal end of the B-chain of human insulin are reversed (human insulin: Pro^{B28}Lys^{B29}; insulin lispro: Lys^{B28}Pro^{B29});

(iii). ‘Insulin glulisine’ differs from human insulin in that the amino acid asparagine at position B3 is replaced by lysine and the lysine in position B29 is replaced by glutamic acid;

(iv). “Insulin glargine” differs from human insulin in that the asparagine at position A21 is replaced by glycine and the B chain is extended at the carboxy terminal by two arginines.

As used herein, the term “amphiphilic” refers to molecules, substances, active pharmaceutical ingredients, structures or part thereof that possess both hydrophobic and hydrophilic properties.

As used herein, the term “aqueous” refers to a solution in which the solvent is water and/or to a suspension in which the external phase is water and/or to an emulsion in which the continuous phase is water.

As used throughout the description and the claims of this specification, the word “comprise” and variations of the word, such as “comprising” and “comprises” is not intended to exclude other additives, components, integers or steps.

As used herein, the term “cationic charged polymer” refers to a polymer, protein, peptide, and/or polyaminoacid which has a pKa and/or an isoelectric point (IEP) above 7, above 8, above 9, above 10, above 11, above 12, above, 13, i.e. the net charge of such polymer, protein, peptide, and/or polyaminoacid is positive at neutral pH (pH 7). Examples of cationic charged polymers include, but are not limited to, the following: protamine; lixisenatide; polyaminoacids wherein more than 50%, more than 55%, more than 60%, more than 65%, more than 70%, more than 75%, more than 80%, more than 85%, more than 90%, more than 91%, more than 92%, more than, 93%, more than 94%, more than 95%, more than 96%, more than 97%, more than 98%, more than 99%, equal to 100% of the monomers are arginine (e.g. polyarginine); polyaminoacids wherein more than 50%, more than 55%, more than 60%, more than 65%, more than 70%, more than 75%, more than 80%, more than 85%, more than 90%, more than 91%, more than 92%, more than, 93%, more than 94%, more than 95%, more than 96%, more than 97%, more than 98%, more than 99%, equal to 100% of the monomers are lysine (e.g. polylysine); polyaminoacids wherein more than 50%, more than 55%, more than 60%, more than 65%, more than 70%, more than 75%, more than 80%, more than 85%, more than 90%, more than 91%, more than 92%, more than, 93%, more than 94%, more than 95%, more than 96%, more than 97%, more than 98%, more than 99%, equal to 100% of the monomers are histidine (e.g. polyhistidine). In one embodiment the active pharmaceutical ingredient and/or an analogue and/or derivative thereof may also be a cationic charged polymer if said active pharmaceutical ingredient and/or an analogue and/or derivative has a pKa and/or an isoelectric point (IEP) above 7, i.e. the net charge is positive. In another embodiment the at least one active pharmaceutical ingredient selected from the group consisting of at least one insulin, insulin analogue, insulin derivative, glucagon-like peptide-1 receptor agonist (GLP1R agonist) and/or dual GLP-1 receptor/glucagon receptor agonist and/or an analogue and/or derivative thereof is a cationic charged polymer. The pKa and/or IEP can either be calculated or measured using methods known in the art.

As used herein, the term “negatively charged polymer” refers to a polymer, polysaccharide, glucosaminoglycan, hydrocolloid, gum, protein, peptide, and/or polyaminoacid, which has a pKa and/or isoelectric point (IEP) of below 7, below 6, below 5, below 4, below 3, below 2—i.e. the net charge of such polymer, polysaccharide, glucosaminoglycan, hydrocolloids, gum, protein, peptide, and/or polyaminoacid is negative at neutral pH (pH 7). Examples of negatively charged polymers include, but are not limited to, the following: polysialic acid, hyaluronic acid, alginic acid, polyglucuronic acid, chondroitin sulphate, heparan sulphate, dextran sulphate, xanthan gum, carrageenan, pectin, gum arabic, gum tragacanth, polyacrylic acid, polyglutamic acid, carboxymethyl cellulose, albumin (pl: 4.7), casein (pl: 4.6), type B gelatin (pl: 4.7-5.2), collagen (pl: 4.7 -5.2), polyaspartic acid, polyaminoacids wherein more than 50%, more than 55%, more than 60%, more than 65%, more than 70%, more than 75%, more than 80%, more than 85%, more than 90%, more than 91%, more than 92%, more than, 93%, more than 94%, more than 95%, more than 96%, more than 97%, more than 98%, more than 99%, equal to 100% of the monomers are glutamic acid; polyaminoacids wherein more than 50%, more than 55%, more than 60%, more than 65%, more than 70%, more than 75%, more than 80%, more than 85%, more than 90%, more than 91%, more than 92%, more than, 93%, more than 94%, more than 95%, more than 96%, more than 97%, more than 98%, more than 99%, equal to 100% of the monomers are aspartic acid.

In one embodiment the active pharmaceutical ingredient and/or an analogue and/or derivative thereof may also be a negatively charged polymer if said active pharmaceutical ingredient and/or an analogue and/or derivative has a pKa and/or an isoelectric point (IEP) below 7, below 6, below 5, below 4, below 3, below 2, i.e. the net charge is negative. In another embodiment the at least one active pharmaceutical ingredient selected from the group consisting of at least one insulin, insulin analogue, insulin derivative, glucagon-like peptide-1 receptor agonist (GLP1R agonist) and/or dual GLP-1 receptor/glucagon receptor agonist and/or an analogue and/or derivative thereof is a negatively charged polymer. The pKa and/or IEP can either be calculated or measured using methods known in the art.

As used herein, the terms “derivative of insulin” and “insulin derivative” refer to a polypeptide which has a molecular structure which formally can be derived from the structure of a naturally occurring insulin, for example that of human insulin, in which one or more organic substituents (e.g. a fatty acid) is bound to one or more of the amino acids. Optionally, one or more amino acids occurring in the naturally occurring insulin may have been deleted and/or replaced by other amino acids, including non-codeable amino acids, or amino acids, including non-codeable, have been added to the naturally occurring insulin. Examples of derivatives of insulin include, but are not limited to, the following:

(i). ‘Insulin detemir’ which differs from human insulin in that the C-terminal threonine in position B30 is removed and a fatty acid residue (myristic acid) is attached to the epsilon-amino function of the lysine in position B29.

(ii). ‘Insulin degludec’ which differs from human insulin in that the last amino acid is deleted from the B-chain and by the addition of a glutamyl link from LysB29 to a hexadecandioic acid.

As used herein, the term “fast acting insulin” or “short acting insulin” refers to insulin analogues and/or insulin derivatives, wherein the insulin-mediated effect begins within 5 to 15 minutes and continues to be active for 3 to 4 hours. Examples of fast acting insulins include, but are not limited to, the following: (i). insulin aspart; (ii). insulin lispro and (iii). insulin glulisine. As used herein, the term “formulation” refers to a product comprising specified ingredients in predetermined amounts or proportions, as well as any product that results, directly or indirectly, from combining specified ingredients in specified amounts. In relation to pharmaceutical formulations, this term encompasses a product comprising one or more active pharmaceutical ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients. In general, pharmaceutical formulations are prepared by uniformly bringing the active pharmaceutical ingredient into association with a liquid carrier or a finely divided solid carrier or both, and then, if necessary, shaping the product into the desired formulation. The pharmaceutical formulation includes enough of the active pharmaceutical ingredient to produce the desired effect upon the progress or condition of diseases. As used herein, the term “formulation” may refer to a solution as well as to a suspension or to an emulsion. As used herein, the terms “formulation” and “composition” are meant to be synonyms, i.e., have identical meaning. The pharmaceutical compositions are made following conventional techniques of pharmaceutical technology involving mixing, filling and dissolving the ingredients, as appropriate, to give the desired oral, parenteral, rectal, transdermal, or topical products.

As used herein, the term “GLP-1 receptor agonist” refers to compounds which have an agonistic activity at the glucagon-like peptide-1 receptor. Examples of GLP-1 receptor agonists include, but are not limited to, the following: exenatide/exendin-4, liraglutide, lixisenatide, dulaglutide, albiglutide, semaglutide, taspoglutide, rExendin-4, CJC-1134-PC, PB-1023, TTP-054, HM-11260C, CM-3, GLP-1 Eligen, ORMD-0901, NN9924 (OG217SC), Nodexen, Viador-GLP-1, CVX-096, ZYOG-1, ZYD-1, MAR-701, ZP-3022, CAM-2036, DA-15864, ARI-2651, ARI-2255, exenatide-XTEN and glucagon-XTEN, AMX-8089+VRS-859 and polymer bound GLP-1 and GLP-1 analogues.

As used herein, the term “dual GLP-1 receptor/glucagon receptor agonist” refers to compounds which have agonistic activity at both the GLP-1 receptor and the glucacon receptor. Examples of dual GLP-1 receptor/glucagon receptor agonist include, but are not limited to, the following: oxyntomodulin, MAR701, MAR-709, and BHM081/BH M089/BHM098, ZP2929, OAP-189, TT-401/402, MOD-6030, LAPS-HMOXM25 (BHM-034).

As used herein, the term “human insulin” refers to the human hormone whose structure and properties are well-known. Human insulin has two polypeptide chains (chains A and B) that are connected by disulphide bridges between cysteine residues, namely the A-chain and the B-chain. The A-chain is a 21 amino acid peptide and the B-chain is a 30 amino acid peptide, the two chains being connected by three disulphide bridges: one between the cysteines in position 6 and 11 of the A-chain; the second between the cysteine in position 7 of the A-chain and the cysteine in position 7 of the B-chain; and the third between the cysteine in position 20 of the A-chain and the cysteine in position 19 of the B-chain.

As used herein, the term “hydrophilic” refers to molecules, substances, active pharmaceutical ingredients, structures or part thereof that are attracted by water and are able to dissolve in water or in polar solvents.

As used herein, the term “including” is used to mean “including but not limited to”. “Including” and “including but not limited to” are used interchangeably.

As used herein the term “Hydrophilic Lipophilic Balance” and/or the abbreviation “HLB” refers to a parameter to characterize and classify surfactants. The hydrophilic-lipophilic balance (HLB) was first defined by Griffin (Griffin, W. C. (1949) J. Soc. Cosmet. Chem. 1:311-26) in 1948 as “the balance of the size and strength of hydrophilic and lipophilic groups that are present in the molecule of an emulsifier”). The scale for HLB is arbitrary. The value of 1 was assigned to the HLB value of pure oleic acid and a value of 20 was assigned to sodium oleate.

The method for calculating HLB as defined in the present invention is Davies' method (Davies, J. T. (1957) “A quantitative kinetic theory of emulsion type. I. Physical chemistry of the emulsifying agent. Gas/Liquid and Liquid/Liquid Interfaces”. Proceedings of 2^{nd} International Congress Surface Activity, Butterworths, London, pp. 426-38) which is based on the functional groups of the molecule under investigation. Each group is given a value, related with their solubility in water (Table 1), the higher the value the more soluble in water. In this way, HLB can be calculated as follows:

HLB=7+m·Hh−n·HI (eq. 1)

Where m is the number of hydrophilic groups, Hh is the value of the hydrophilic groups, n is the number of lipophilic groups and HI is their value.

TABLE 1
Group numbers of different functional
groups presented in emulsifiers

| | Group Number |
|---|---|
| Hydrophilic groups | |
| —SO₄⁻Na⁺ | 38.7 |
| —COO⁻K⁺ | 21.1 |
| —COO⁻Na⁺ | 19.1 |
| N (tertiary amine) | 9.4 |
| Ester (sorbitan ring) | 6.8 |
| Ester (free) | 2.4 |
| —COOH | 2.1 |
| Hydroxyl (free) | 1.9 |
| —O— | 1.3 |
| Hydroxyl (sorbitan ring) | 0.5 |
| Lipohilic groups | |
| —CH— | −0.475 |
| —CH₂— | |
| CH₃— | |
| ═CH— | |

For specific families of emulsifiers, equation 1 can be simplified. For instance, for the polyoxyethylene alkyl ethers and polyoxyethylene ester, the ethylene oxide is the hydrophilic group. The HLB value of such surfactants, according to Griffin, is equivalent to the mass percentage of oxyethylene content (E) divided by 5.

HLB=E/5 (eq. 2)

For a mixture of emulsifiers, HLB values are obtained by applying the additive property:

HLB=ΣHLBᵢ·fᵢ (eq. 3)

Where fi is the mass fraction of the surfactant i

Apart from emulsifiers, oils can be assigned a HLB value. In this case, the HLB of the oil is the

HLB of an emulsifier or mixture of emulsifiers that produces the more stable emulsion. Regarding this, Robbers & Bhatia (Robbers, J. E. & V. N. Bhatia (1961) J. Pharm. Sci. 50: 708-9) developed an experimental technique for the rapid determination of the HLB of an oil. In this method, series of emulsions are prepared covering the range of HLB values between two stocks emulsions. The desired amounts of each stock emulsion are weighted into a 15 mL graduated centrifuge tube to make a total of 10 g of mixed emulsion. Then, the tubes are centrifuged at 1500 rpm. The HLB value of the emulsifiers in the emulsion showing the least separation is taken to be equal to the required HLB value for the oil.

Once the HLB of an oil is known, it can be used to determine the HLB of an emulsifier. In this case it is necessary to use a mixture of emulsifiers, one with a known HLB and the other for the unknown HLB. Different mixtures with different proportions of the emulsifiers are prepared. The one that lead to the most stable emulsion obey the expression

HLBₒᵢₗ=x·HLB1+(100−x)·HLB2 (eq. 4)

Where HLBoil and HLB2 are known, x is calculated experimentally and consequently HLB1 can be calculated from equation 4.

Chun & Martin (Chun, A. H. C. & A. N. Martin (1961) J. Pharm.Sci. 50: 732-6) developed an interfacial tension method for the evaluation of HLB values of water soluble surface-active agents. One-tenth percent aqueous solutions of surfactant were over layered with toluene and the interfacial tension were measured. A linear relationship resulted when the interfacial tension values were plotted against HLB values.

Huebner (Huebner, V. H., (1962) Anal. Chem. 34: 488-91) developed a rapid method of determining relative polarity of surfactants. This method is based on a comparison of the retention times of methanol and n-hydrocarbons when a surfactant is used as the liquid phase in a gas-liquid chromatography column.

Gorman & Hall (Gorman, W. G. & G. D. Hall (1963) J. Pharm.Sci. 52: 442-6) found a linear relationship with the log dielectric constant of surfactants and its HLB values.

Ben-Et & Tatarsky (Ben-Et, G. & D. Tatarsky (1972) J. Am. Oil Chem. 49: 499-500) determined HLB values for various commercial non-ionic surfactants by means of high resolution NMR.

As used herein, the term “kit” refers to a product (e.g. medicament, kit-of-parts) comprising one package or one or more separate packages of:

(i). A pharmaceutical formulation containing an active pharmaceutical ingredient and at least one further active pharmaceutical ingredient and optionally a medical device. The at least one further active pharmaceutical ingredient may be present in said pharmaceutical formulation, i.e. the kit may comprise one or more packages, wherein each package comprises one pharmaceutical formulation which comprises two or more active pharmaceutical ingredients.

The further active pharmaceutical ingredient may also be present in a further pharmaceutical formulation, i.e. the kit may comprise separate packages of two or more pharmaceutical formulations, wherein each pharmaceutical formulation contain one active pharmaceutical ingredient; or (ii). a pharmaceutical formulation containing an active pharmaceutical ingredient and medical device. A kit may comprise one package only or may comprise one or more separate packages. For example, the kit may be a product (e.g. medicament) containing two or more vials each containing a defined pharmaceutical formulation, wherein each pharmaceutical formulation contains at least one active pharmaceutical ingredient. For example, the kit may comprise (i.) a vial containing a defined pharmaceutical formulation and (ii). further a tablet, capsule, powder or any other oral dosage form which contains at least one further active pharmaceutical ingredient. The kit may further comprise a package leaflet with instructions for how to administer the pharmaceutical formulation and the at least one further active pharmaceutical ingredient.

As used herein, the term “lipophilic” refers to molecules, substances, active principles, structures or part thereof that are not able to interact on their own with water molecules and are primarily dissolved in apolar solvents.

As used herein, the term “long acting insulin” refers to insulin analogues and/or insulin derivatives, wherein the insulin-mediated effect begins within 0.5 to 2 hours and continues to be active for about or more than 24 hours. Examples of fast acting insulins include, but are not limited to, the following: (i). insulin glargin; (ii). insuline detemir and (iii). insulin degludec.

As used herein, the term “medical device” means any instrument, apparatus, implant, in vitro reagent or similar or related article that is used to diagnose, prevent, or treat a disease of other condition, and does not achieve its purpose through pharmacological action within or on the body. As used herein, a medical device may be a syringe, an insulin injection system, an insulin infusion system, an insulin pump or an insulin pen injection.

As used herein, the term “nanometric size”, “nanocapsule” or “nanocapsule system” refers to a structures having an average diameter of less than 1 μm, and therefore comply with the definition of nanosystems: colloidal system constituted on the basis of polymers with a size less than 1 μm, i.e. they have a size between 1 and 999 nm, in one embodiment between 30 and 500 nm. Average diameter means that measured by the Dynamic Light Scattering (DLS) technique, which is defined by the hydrodynamic diameter of a sphere that diffuses at the same rate as the particles that are being measured. The size of the nanocapsules or the nanocapsule system is influenced mainly by the composition and the conditions of formation and can be measured using standard methods known by a person skilled in the art and which are described in the examples section. In this connection, as can be verified, their size does not change markedly on altering the proportion of coating compound in the formulation, systems of nanometric size being obtained in all cases.

As used herein, unless specifically indicated otherwise, the conjunction “or” is used in the inclusive sense of “and/or” and not the exclusive sense of “either/or”.

As used herein, the term “pharmaceutical” refers to the intended use in the medical diagnosis, cure, treatment and/or prevention of diseases.

As used herein, the term “pharmaceutically acceptable” refers to physiologically well tolerated by a mammal or a human.

As used herein, the term “polyarginine” refers to a peptide or polyaminoacid with a molecular weight in the range of 1-300KDa, with a number of arginine monomers between 8 and 200, where the number of arginine monomers represents at least 50%, at least 55%, at least 60%, at least 65%, at least 70%, at least 75%, at least 80%, at least 85%, at least 90%, at least 95%, at least 96%, at least 97%, at least 98%, at least 99% or 100% of the total amount of monomers.

As used herein, the term “polyethylene glycol” refers to a polyether compound having the following structure: H[O—CH₂—CH₂)ₙ—OH. As used herein, the terms “polyethylene glycol”, “polyethylene oxide (PEO)”, and “polyoxyethylene (POE)” are meant to be synonyms, i.e., have identical meaning

As used herein, the term “polylysine” refers to a peptide or polyaminoacid with a molecular weight in the range of 1-300KDa, with a number of lysine monomers between 8 and 200, where the number of lysine monomers represents at least 50%, at least 55%, at least 60%, at least 65%, at least 70%, at least 75%, at least 80%, at least 85%, at least 90%, at least 95%, at least 96%, at least 97%, at least 98%, at least 99% or 100% of the total amount of monomers.

As used herein, the term “protamine” refers to a mixture of strongly basic peptides which belongs to a family of natural arginine-rich polypeptides. It was originally isolated from the sperm of salmon and other species of fish but is now produced primarily recombinant through biotechnology. It contains approximately 70% of L-arginine monomers and has a molecular weight of 4000-10000 Da. As protamine contains amino acids having free basic side chains, it has a certain buffering capacity and is therefore considered to be a buffering agent. Protamine may be used as protamine sulfate or protamine hydrochloride. Protamine has been approved by the regulatory authorities as a pharmaceutical excipient and nowadays its main application is in formulations for sustained release of insulin: NPH (Neutral Protamine Hagedorn), as well as possessing marketing authorization as an active pharmaceutical ingredient, as the antidote for heparin poisoning. As used herein, term “protamine” includes the water-soluble salts of protamine as well as water-soluble protamine derivatives.

As used herein, the term “surfactant” refers to a component that possesses structures and/or functional groups that allow them to interact simultaneously with the lipophilic and hydrophilic part of the formulation. Examples of surfactants include, but are not limited to, the following:

polyoxyethylene sorbitan monooleate (polysorbate 80; Tween 80®; HLB 15), polyoxyethylene sorbitan monolaurate (polysorbate 20; Tween 20®; HLB 16.7), polyoxyethylene sorbitan monostearate (Tween® 60, HLB 14.9 and Tween 61®; HLB 9.6), polyoxyethylene sorbitan monooleate (Tween 81®; HLB 10), polyoxyethylene sorbitan tristearate (Tween 65®; HLB 10.5), polyoxyethylene sorbitan trioleate (Tween 85®; HLB 11), polyoxyethylene sorbitan monolaurate (Tween® 20, HLB 16.7 and Tween 21®; HLB 13.3); PEGylated fatty acid esters and mixtures with PEG, polyethylene glycol monostearate (HLB 11.6), polyethylene glycol stearate, polyethylene glycol stearate 40 (HLB 17), polyethylene glycol dilaurate 400 (HLB 9.7), polyethylene glycol dilaurate 200 (HLB 5.9), polyethylene glycol monopalmitate (HLB 11.6), polyethylene glycol stearate, polyethylene glycol stearate 40 (HLB 16.9) polyethylene glycol stearate 100 (HLB 18.8), Solutol HS15® (HLB 15), polyethylene glycol-15-hydroxystearate (HLB 14-16), D-alpha-tocopheryl polyethylene glycol succinate (TPGS; HLB 13.2), triethanolammonium oleate (HLB 12), sodium oleate (HLB 18), sodium cholate (HLB 18), sodium deoxycholate (HLB 16), sodium lauryl sulphate (HLB 40), sodium glycocholate (HLB 16-18), triethanolamine oleate (HLB 12), gum tragacanth (HLB 11.9) and sodium dodecyl sulphate (HLB 40); Poloxamer 124 (HLB 16), Poloxamer 188 (HLB 29), Poloxamer 237 (HLB 29), Poloxamer 238 (HLB 28), Poloxamer 278 (HLB 28), Poloxamer 338 (HLB 27), and Poloxamer 407 (HLB 22), sorbitan monooleate (Span® 80, HLB 4.3), sorbitan monolaurate (Span® 20, HLB 8.6), sorbitan monostearate (Span® 60, HLB 4.7), sorbitan trioleate (Span® 85, HLB 1.8), sorbitan sesquiolate (Span® 83, HLB 3.7), sorbitan monopalmitate (Span® 40, HLB 6.7), sorbitan isostearate (Span® 120, HLB 4.7), polyoxyethylene sorbitan monopalmitate (Tween® 40, HLB 15.6) or any combination thereof.

As used herein, the term “treatment” refers to any treatment of a mammalian, for example human condition or disease, and includes: (1) inhibiting the disease or condition, i.e., arresting its development, (2) relieving the disease or condition, i.e., causing the condition to regress, or (3) stopping the symptoms of the disease.

Concentrations, amounts, solubilities, particle size, wavelength, pH values, weight mass, molecular weight, percent and other numerical date may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited.

Further embodiments of the present invention include the following:

In one aspect, the present invention provides a nanocapsule system that comprises:
a. a surface layer that comprises a cationic charged polymer; and
b. optionally a second surface layer that comprises a negatively charged polymer; and
c. a lipid core that comprises at least one lipophilic compound;
d. at least one surfactant or a mixture of surfactants; and
e. at least one active pharmaceutical ingredient selected from the group consisting of insulin, insulin analogue, insulin derivative, glucagon-like peptide-1 receptor agonist (GLP1R agonist) dual GLP-1 receptor/glucagon receptor agonist and any combination thereof.

In one aspect, the nanocapsule system of the present invention comprises a cationic charged polymer which is selected from the group consisting of peptides, polyaminoacids, polylysine, polyarginine, lixisenatide, and protamine.

In one aspect, the nanocapsule system of the present invention comprises a negatively charged polymer which is selected from the group consisting of polysialic acid, polyacrylic acid, hyaluronic acid, polyglutamic acid, alginic acid, polyglucuronic acid, and xanthan gum.

In one aspect, the nanocapsule system of the present invention comprises a surfactant or a mixture of surfactants which is characterized by possessing a hydrophilic-lipophilic balance (HLB) above 8.

In one aspect, the nanocapsule system of the present invention comprises a surfactant or a mixture of surfactants which comprises at least one surfactant selected from the group consisting of sorbitan esterified with at least one fatty acid and ethoxylates thereof, fatty acid esters, fatty acid salts, gum tragacanth, bile salts and bile salt derivatives and poloxamers.

In one aspect, the nanocapsule system of the present invention comprises at least one surfactant which is a sorbitan esterified with at least fatty acid or ethoxylates thereof selected from the group consisting of sorbitan monooleate, sorbitan monolaurate, sorbitan monostearate, sorbitan trioleate, sorbitan sesquiolate, sorbitan monopalmitate, sorbitan isostearate, polyoxyethylene sorbitan monooleate (polysorbate 80), polyoxyethylene sorbitan monolaurate (polysorbate 20), polyoxyethylene sorbitan monostearate, polyoxyethylene sorbitan monooleate, polyoxyethylene sorbitan tristearate, polyoxyethylene sorbitan trioleate, polyoxyethylene sorbitan monolaurate, polyoxyethylene sorbitan monopalmitate and any combination thereof In one aspect, the nanocapsule system of the present invention comprises at least one surfactant which is a fatty acid ester or a fatty acid salt selected from the group consisting of polyethylene glycol monostearate, polyethylene glycol stearate 40, polyethylene glycol dilaurate, polyethylene glycol monopalmitate, polyethylene glycol stearate 100, polyethylene glycol-15-hydroxystearate, D-alpha-tocopheryl polyethylene glycol succinate (TPGS), triethanolammonium oleate, sodium oleate, sodium lauryl sulphate, triethanolamine oleate, and sodium dodecyl sulfate and any combination thereof.

In one aspect, the nanocapsule system according to the present invention comprises at least one surfactant a mixture of surfactants or at least one surfactant, which is a bile salt or bile salt derivative selected from the group consisting of sodium cholate (HLB 18), sodium deoxycholate (HLB 16), sodium glycocholate (HLB 16-18), sodium taurocholate (HLB 16), sodium taurodeoxycholate (HLB 20.1).

In one aspect, the nanocapsule system of the present invention comprises at least one surfactant which is a poloxamer selected from the group consisting of Poloxamer 124, Poloxamer 188, Poloxamer 237, Poloxamer 238 (HLB 28), Poloxamer 278 (HLB 28), Poloxamer 338, Poloxamer 407, and any combination thereof.

In one aspect, the nanocapsule system of the present invention comprises at least one surfactant which is selected from the group consisting of sodium cholate, sodium glycocholate, sodium deoxycholate, polyethylene glycol stearate, polyethylene glycol-15-hydroxystearate, D-alpha-tocopheryl polyethylene glycol succinate (TPGS), polyoxyethylene sorbitan monooleate, polyoxyethylene sorbitan monolaurate, Poloxamer, and any combination thereof.

In one aspect, the nanocapsule system of the present invention comprises a lipophilic compound which is selected from the group consisting of peanut oil, cottonseed oil, olive oil, castor oil, soybean oil, safflower oil, geranium oil, palm oil, alpha-tocopherol (vitamin E), oleic acid, linoleic acid, isopropyl myristate, squalene, caprylic/capric triglyceride, linoleoyl macrogol-6 glycerides (corn oil PEG-6 esters), triglycerides medium chain, glyceryl oleate, glyceryl linoleate, glycerol monooleate, and any combination thereof.

In one aspect, the nanocapsule system of the present invention comprises an insulin which is human insulin.

In one aspect, the nanocapsule system of the present invention comprises an insulin analogue which is selected from the group consisting of insulin aspart, insulin lispro, insulin glargine and insulin glulisine or any combinations thereof.

In one aspect, the nanocapsule system of the present invention comprises an insulin derivative which is selected from the group consisting of insulin detemir and insulin degludec.

In one aspect, the nanocapsule system of the present invention comprises a glucagon-like peptide-1 receptor agonist (GLP1R agonist) which is selected from the group consisting of exendin-4, liraglutide, lixisenatide, dulaglutide, albiglutide, semaglutide, taspoglutide, and any combinations thereof.

In one aspect, the present invention provides a pharmaceutical composition that comprises the nanocapsule system of the present invention and optionally one or more further active pharmaceutical ingredients and optionally one or more pharmaceutically acceptable excipients.

In one aspect, the nanocapsule system of the present invention and/or the pharmaceutical composition of the present invention is characterized in that it is lyophilized.

In one aspect, the present invention provides a kit comprising one or more separate packages of
a. the nanocapsule system of the present invention or the pharmaceutical composition of the present invention; and
b. at least one further active pharmaceutical ingredient; and optionally
c. a medical device.

In one aspect, the nanocapsule system of the present invention and/or the pharmaceutical composition of the present invention and/or the kit of the present invention is
a. for use in the treatment of diabetes mellitus; and/or
b. for use in the treatment of hyperglycemia; and/or
c. for use in lowering blood glucose level.

In one aspect, the nanocapsule system of the present invention and/or the pharmaceutical composition of the present invention and/or the kit of the present invention is for subcutaneous, transdermal, buccal, oral, pulmonary or nasal administration.

In one aspect, the present invention provides a method of treating diabetes mellitus and/or a method of treating hyperglycemia and/or a method of lowering blood glucose levels in a subject in need thereof comprising administering the nanocapsule system of the present invention or the pharmaceutical composition of the present invention.

In one aspect, the present invention provides a medical device for administering the nanocapsule system of the present invention and/or the pharmaceutical composition of the present invention.

In one aspect, the present invention provides a method for producing the nanocapsule system of the present invention comprising the emulsification of an internal lipophilic phase containing the lipophilic compound and an external water phase containing the cationic charged polymer, optionally a negatively charged polymer, and the surfactant or the mixture of surfactants, and the at least one active pharmaceutical ingredient selected from the group consisting of human insulin, insulin analogue, insulin derivative, glucagon-like peptide-1 receptor agonist (GLP1R agonist), dual GLP-1 receptor/glucagon receptor agonist, and any combination thereof.

In one aspect, the method for producing the nanocapsule system of the present invention is a method wherein the at least one active pharmaceutical ingredient selected from the group consisting of human insulin, insulin analogue, insulin derivative, glucagon-like peptide-1 receptor agonist (GLP1R agonist), dual GLP-1 receptor/glucagon receptor agonist, and any combination thereof, is dispersed in the lipophilic phase either as a powder or as an aqueous solution.

In one aspect, the method for producing the nanocapsule system of the present invention is a method, wherein the at least one active pharmaceutical ingredient selected from the group consisting of human insulin, insulin analogue, insulin derivative, glucagon-like peptide-1 receptor agonist (GLP1R agonist), dual GLP-1 receptor/glucagon receptor agonist, and any combination thereof, is dispersed either in the lipophilic phase either as a powder or as an aqueous solution.

### BRIEF DESCRIPTION OF THE FIGURES

FIG. 1: A—Colloidal stability of blank protamine nanocapsules in SIF and B—in SIF supplemented with 1% pancreatin at pH 6.8, 37° C.

FIG. 2: The in vitro release profile of insulin glulisine-loaded protamine nanocapsules (Pr-NC: ♦) and polysialic acid-coated protamine nanocapsules (PSA-Pr NC: ▪) in SIF, pH 6.8, 37° C.

FIG. 3: Colloidal stability of insulin glulisine-loaded protamine nanocapsules in FaSSIF pH 6.5, 37° C.

FIG. 4: An example of size distribution curves following the incubation of insulin glulisine-loaded protamine nanocapsules in updated simulated intestinal fluid in a fed state (FeSSIF V2). A: FeSSIF-V2 only. B and C: insulin glulisine-loaded protamine nanocapsules after incubation in FeSSIF-V2 at T0 and T4, respectively.

FIG. 5: A: Colloidal stability of insulin glulisine-loaded protamine nanocapsules in FeSSIF V2 at pH 5.8, 37° C. (attenuator fixed at 6, n=3); B: Colloidal stability of insulin glulisine-loaded protamine nanocapsules post-coated with polysialic acid in FeSSIF V2 at pH 5.8, 37° C. (attenuator fixed at 6, n=3)

FIG. 6: Proteolysis of insulin glulisine encapsulated in protamine nanocapsules upon incubation in FeSSIF V2 at pH 5.8, 37 ° C. . A: Original prototypes (protamine nanocapsules with no polysialic acid coating) and B prototypes post-coated with polysialic acid.

FIG. 7: In vitro release profile of insulin glulisine from insulin glulisine-loaded protamine nanocapsules (Pr-NC: ♦) and polysialic acid-coated protamine nanocapsules (PSA-Pr NC: ▪) in Fassif v2, pH 6.5, 37° C.

FIG. 8: Physico-chemical characteristics of insulin glulisine-loaded protamine nanocapsules before and after freeze drying.

FIG. 9: Evolution of blood glucose levels upon the intraduodenal administration of glulisine-loaded protamine nanocapsules (Pr-NC:▪), polysialic acid-coated protamine nanocapsules (PSA-Pr NC: ▪) or glulisine solution (x) to canulated normoglycemic rats. (single dose of 50 IU/kg, the number of animals treated varied between 5 and 12, and they were grouped from different experiments)

FIG. 10: Influence of the final formulation pH on particle size (A), Z-potential (B) and EE (C) of Parg NC.

FIG. 11: Colloidal stability (particle size and count rate) of the insulin loaded non-buffered PArg NC. The attenuator of DLS is fixed to 6.

FIG. 12: Colloidal stability (particle size and count rate) of the insulin loaded 20 mM acetate buffer based PArg NC. The attenuator of DLS is fixed to 6.

FIG. 13: Colloidal stability (particle size and count rate) of PArg nanocapsules (formulation E in table 5) in SIF (pH 6.8), FaSSIF-V2, (pH 6.5) and FeSSIF-V2 (pH 5.8)

FIG. 14: The in vitro insulin release profile of 20mM acetate buffer based NCs in FaSSIF-V2 medium. The amount of insulin released to the media (A) and that remained in the NC cream (B).

### EXAMPLES

The meaning of the abbreviations used throughout the examples is presented below.
1. Pr: Protamine as defined herein; the salt used in the following examples was protamine sulfate (Yuki Gosei Kogyo Co., Ltd.). Another protamine salt (SIGMA) was used, without significant differences arising for any of the examples.
2. PArg: Polyarginine. Polyarginine used in the following examples is of different molecular weight (one chloride salt of 26-37kDa from PTS, Spain and another chloride salt of 5-15kDa, Sigma-Aldrich).
3. PEG: polyethylene glycol. PEG stearate with two different PEG molecular weights have been used: Simulsol® M52 with a PEG chain of 2KDa and Simulsol® M59 with a PEG chain of 5 KDa (Seppic).
4. Nanoemulsion (NE): This term is used for simplicity in the examples to refer to an emulsion comprising an oil and one or more suitable surfactants, with a size below 1 micron. The only difference from the nanocapsules is the absence of protamine/polyarginine in the surface of the systems.
5. Nanocapsules (NCs): This term is used for simplicity in the examples and the figures to refer to the nanosystems (size below 1 micron) comprising a lipid core, one or more suitable surfactants and a polymer and one or two polymer layers around the oily core.

### Example 1

Example 1 relates to the influence of the composition of protamine nanocapsules on their physicochemical properties insulin loading capacity, insulin stability and release profile. Insulin encapsulation capacity was evaluated using glulisine and Zn-hexameric insulin (human insulin) provided by Sanofi-Aventis Deutschland GmbH.

### Materials and Methods

Protamine sulfate used in this work was purchased from Yuki Gosei Kogyo, Ltd. (Japan). The stabilizing surfactants, polyethylene glycol PEG-Stearate 40 and 100 were obtained from Seppic (France) or CRODA. Caprylic/capric triglyceride (Miglyol® 812) was provided by Sasol Germany GmbH (Germany) or Cremer Oleo Division and oleic acid was purchased from Sigma-Aldrich, Spain. Colominic acid sodium salt (polysialic acid) was purchased from NACALAI TESQUE, INC, Japan. Insulin glulisine and human insulin were obtained from Sanofi. Sodium cholate, sodium glycocholate were purchased from Sigma-Aldrich or DEXTRA, pancreatin 4 x USP, trehalose, sucrose and Triton-X100, were purchased from Sigma-Aldrich (Spain). All other products used were of high purity or reagent grade. Blank protamine nanocapsules were prepared by the solvent displacement technique as follows: 12 mg PEG-stearate and 10 mg sodium cholate were dissolved in 750 pl of ethanol, followed by the addition of the oil, Miglyol® or oleic acid 62.5 μL and 4.25 ml of acetone. This organic phase was immediately poured over 10 ml of an aqueous phase with 0.05% w/v protamine. The elimination of organic solvents was performed by evaporation under vacuum (Rotavapor Heidolph, Germany), to obtain a nanocapsule formulation with a constant volume of 5 ml.

Insulin-loaded (either insulin glulisine or human insulin) protamine nanocapsules were prepared following the same procedure as in blank nanocapsules, where the organic phase was prepared by combining all the lipidic components dissolved in organic solvents (ethanol and acetone). A low volume (0.05 ml) of a concentrated insulin solution (30 mg/ml) was added into the lipid phase before transferring it to the aqueous phase. Insulin solutions (containing either insulin glulisine or human insulin) of different pH values were prepared by adding either 0.01 M HCl or 0.01M NaOH.

Protamine nanocapsules were isolated by ultracentrifugation (Optima™ L-90K, Ultracentrifuge, Beckman Coulter, USA) at 30000 RPM for 1 h (at 15° C.) and resuspended in ultrapure water to the initial volume or concentration of protamine.

In the case of specific prototypes and in order to increase their stability upon contact with pancreatic enzymes, protamine nanocapsules were provided with an additional coating layer consisting of polysialic acid. For this, a volume of 0.5 ml, protamine nanocapsules (concentration 16.3 mg/ml) was incubated with a volume of 0.1 ml solution of polysialic acid at a concentration 1 mg/ml. The final nanocapsules: polysialic acid ratio was of 8.15:0.1 w/w). The concentration of nanocapsules was measured taking into account all components in the formulation.

The average diameter and polydispersity index (PDI) of protamine nanocapsules were measured by dynamic light scattering on a zeta sizer Nano series DTS 1060 (Malvern instruments), after dilution with ultrapure water. The zeta potential was measured by laser-Doppler anemometry after diluting the samples with KCl 1 mM (Zetasizer®, NanoZS, Malvern Instruments, Malvern, UK).

The stability of protamine nanocapsules upon incubation at 37° C. in different media was studied. The media were: Simulated intestinal fluid (SIF) pH 6.8 in the presence and absence of enzymes, fasted state, upper small intestine (FaSSIF-V2) pH 6.5 and fed state, upper small intestine (FeSSIF-V2) pH 5.8. Samples were collected at times 0, 0.5, 1, 2 and 4 hours. The SIF with pancreatin and FeSSIF-V2 samples were centrifuged at 5000 x g for 5 minutes to eliminate aggregates of pancreatin in the medium. The size distributions, polydispersity index (PDI) and count rate of the nanocapsules were monitored by dynamic light scattering on a zeta sizer Nano series DTS 1060 (Malvern instruments).

The colloidal stability of the blank nanocapsules was followed during a period of 6 months at different temperatures (4° C., 25° C. and 37° C.) and RH conditions, as recommended by ICH guidelines. Samples of three different batches were withdrawn at predetermined time intervals, determining particle size and zeta potential and describing the suspension appearance to ensure continued viability.

The theoretical % insulin loading was calculated with regard to the mass of insulin (insulin glulisine or human insulin) and all components added during the formulation of the nanocapsules (equation 1). The encapsulation efficiency of insulin (%) was determined using the direct method, which involves the extraction of insulin from the nanocapsules. Insulin loaded nanocapsules (0.1 ml) were digested using a combination of acetonitrile (0.16 ml), 0.1% TFA (0.64 ml) and Triton-X100 (0.1 ml). Then the formulation was vortexed at a high speed to obtain a clear aliquot. The concentration of insulin in this aliquot was determined by reverse phase

HPLC. Finally the loading capacity and % yield of protamine nanocapsules were calculated using equation 3 and 4 respectively:

$$\begin{matrix}Theoretical loading [ \% ] = \frac{Weight of insulin}{\begin{matrix}Weight of all components in \\ nanocapsules + insulin\end{matrix}} 100 & Equation 1 \\ Entrapment efficiency ( EE ) [ \% ] = \frac{Amount of insulin in destructed nanocapsules}{Amount of insulin added} 100 & Equation 2 \\ Loading capacity [ \% ] = \frac{Theoretical mass of insulin EE}{Actual mass of nanocapsules} 100 & Equation 3 \\ \% Yield of nanocapsules = \frac{Actual weight of nanocapsules}{\begin{matrix}Theoretical weight of all \\ components in nanocapsules\end{matrix}} 100 & Equation 4\end{matrix}$$

The in vitro release of insulin from the nanocapsules was determined upon incubation in simulated intestinal fluid (SIF, pH 6.8), and shaken at 100 rpm at 37° C. At specified time intervals (0, 0.25, 0.5, 1, 3 and 6 hours), the supernatant was collected by centrifugation. The concentrations of insulin in the supernatant were determined by reverse phase HPLC method, and the total amount of insulin released from the nanocapsules was calculated.

Blank and insulin glulisine-loaded protamine nanocapsules (1% w/v) were lyophilized (Labconco Corp, USA) in presence/absence of trehalose or sucrose at 5% (w/v). The freeze-dried formulations were resuspended with ultrapure water by manual resuspension and their physicochemical characteristics were evaluated.

### Results

Monodisperse suspensions of protamine nanocapsules with diameters in the 200-250 or 50-500 nm range were prepared, with a polydispersity index (PDI) below 0.3. The zeta potential of these nanocapsules was +12 mV/+30 mV depending on the type and amount of polyethylene glycol (PEG) stearate used (PEG 40 vs. PEG 100). This, as compared to the corresponding negatively charged nanoemulsion, indicates the presence of the protamine shell around the lipid core.

PEG stearate, has been shown to be essential in the development of protamine nanocapsules. This polymer is known for its advantages of improving the stability of nanocarriers in biological media and avoiding unspecific opsonisation. PEG coating has also been shown to give nanocarriers a slippery surface and promote diffusion. For these reasons, we evaluated the influence of the PEG stearate molecular weight (MW) on the physico-chemical characteristics of protamine nanocapsules mainly stability and mucodiffusion. PEG stearate 40 (Simusol M52) molecular weight 2 kDa and PEG stearate 100 (Simusol M59) molecular weight 5 kDa, were compared.

TABLE 2
The physico-chemical properties of the nanoemulsion
(NE) and protamine nanocapsules (NC).

| | | Size | | Z-potential |
|---|---|---|---|---|
| Oil core | Formulation | (nm) | PI | (mV) |
| Miglyol | NE PEG 40 | 195 ± 16 | 0.1 | −13 ± 2 |
| | NE PEG 100 | 222 ± 3 | 0.2 | −32 ± 1 |
| | NC-PR PEG 40 | 210 ± 25 | 0.1 | +12 ± 5 |
| | NC-PR PEG 100 | 238 ± 16 | 0.2 | +30 ± 2 |
| | NC-PR PEG 40 + polysialic acid | 253 ± 12 | 0.2 | −28 ± 3 |
| Oleic | NE PEG 40 | 178 ± 3 | 0.1 | −48 ± 2 |
| acid | NC-PR PEG 40 | 188 ± 6 | 0.1 | −30 ± 8 |

Constant parameters: cholic acid salt (e.g. sodium cholate), PEG-st 40/100 and Miglyol 812 ®

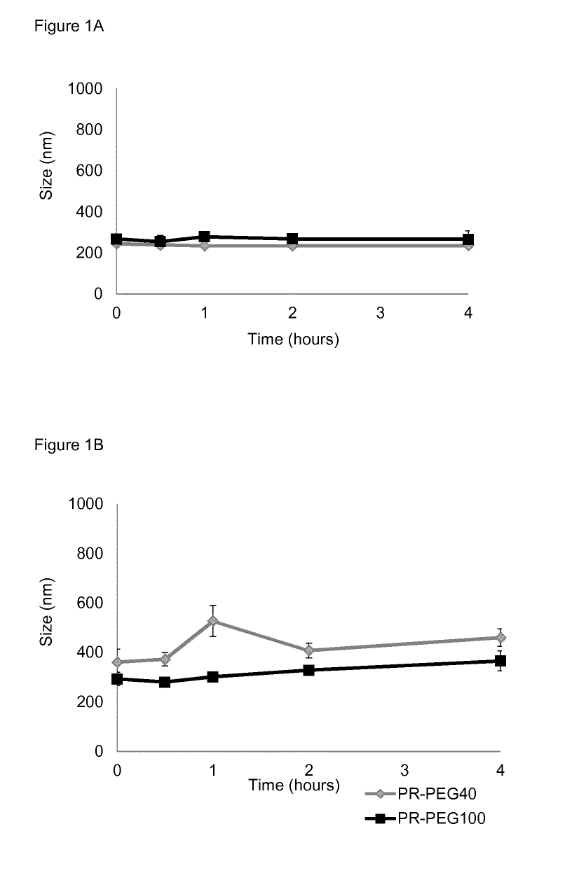

Biological stability of the nanocarrier refers to the stability upon exposure to biological media. Different media of simulated intestinal fluid have been described in literature and used to evaluate the stability of colloidal carriers. In this study, the most relevant media were chosen and compared. Protamine nanocapsules (PR-PEG40 and PR-PEG100) were first incubated in the simple simulated intestinal fluid (SIF) in the presence or absence of pancreatic enzymes at 37° C., pH 6.8 for at least 4 hours. The size and count rate of the nanocapsules did not change during the period of the study (FIG. 1). This implied that the protamine nanocapsules were stable in SIF in the presence or absence of enzymes.

In general PEG 40 seemed to confer more stability to the system as compared to PEG 100, especially when the protamine nanocapsules were incubated in SIF supplemented with pancreatic enzymes. The slight increase in size observed when nanocapsules were incubated in SIF supplemented with pancreatic enzymes might suggest that the enzymes adhere to the surface of the nanocapsules (FIG. 1B). This enzyme-adhesion process could also be supported by the change of nanocapsules zeta potential from positive to negative in the presence of enzymes. This phenomenon should be further corroborated.

Nanocapsules stored at room temperature 25° C. and at 37° C. were stable for at least up to 7 days. Whilst, nanocapsules stored at 4° C. were stable for 6 months without significant changes in their physicochemical characteristics.

The size, zeta potential and mucodiffusion of the protamine nanocapsules were affected by type of PEG-stearate added to the formulation, however, the insulin entrapment efficiency was not affected by this parameter. Moreover, after observing that PR-PEG100 nanocapsules are almost completely immobilised in porcine mucus we focused more on the encapsulation of insulin in PR-PEG40 nanocapsules. Therefore from here onwards, we report on PR-PEG40 nanocapsules.

The pH of the insulin dissolution medium greatly influenced the entrapment efficiency. The highest EE % (60%) was obtained when insulin was dissolved in 0.01 M NaOH, pH 11.4 (Table 3). This could be attributed to the fact that at this pH insulin has a net negative charge and may interact with protamine leading to a higher EE %. The loading capacity and % yield of the nanocapsules at the 60% EE were 2.1% and 38% respectively.

The nanocapsules were prepared containing bile salts and also with an extra coating layer consisting of polysialic acid. These optimization approaches were intended to improve the stability of the nanocapsules especially in more complex media (FeSSIF v2) and to enhance the penetration of the associated peptide.

The results in Table 3a, show that the presence of sodium cholate in the organic phase influences negatively the loading of insulin glulisine whilst the presence of sodium glycocholate resulted in an acceptable encapsulation efficiency.

TABLE 3a
Physico-chemical properties of insulin glulisine- and human insulin-loaded
protamine nanocapsules with a Miglyol ® oil core

| Insulin | | | pH of | Size | | ζ-pot | | Stability at |
|---|---|---|---|---|---|---|---|---|
| type | Formulation | | insulin | (nm) | PDI | (mV) | AE % | 4° C. |
| Glulisine | NC without | A | 11.4 | 425 ± 82 | 0.3 | +2 ± 1 | 66 ± 9 | >1 month |
| | cholate | | | | | | | |
| | NC with Na | B | 2.0 | 266 ± 75 | 0.2 | +18 ± 3 | 38 ± 4 | 6 months |
| | cholate | | | | | | | |
| | NC with Na | C | 2.0 | 382 ± 29 | 0.3 | +6 ± 3 | 62 ± 16 | >1 month |
| | glycocholate | | | | | | | |
| Human | NC without | D | 11.4 | 324 ± 4 | 0.2 | +10 ± 2 | 27 ± 11 | >1 month |
| insulin | cholate | | | | | | | |
| | NC with Na | E | 2.0 | 393 ± 4 | 0.2 | −2 ± 1 | 58 ± 6 | 6 months |
| | cholate | | | | | | | |
| | NC with Na | F | 2.0 | 341 ± 15 | 0.2 | +3 | 32 ± 9 | >1 month |
| | glycocholate | | | | | | | |

Constant parameters, concentration in the final formulation (mg/ml): Miglyol ® = 11.8; PEGst 40 = 2.4; Protamine = 1, (pH of final formulations 6-7).

Moreover, insulin glulisine-loaded nanocapsules were post-coated with polysialic acid to improve the stability of the nanocapsules in the presence of enzymes by avoiding the attachment of enzymes on the surface of the nanocapsules. Consequently, the presence of polysialic acid coating around the nanocapsules led to a change in the zeta potential of the nanocapsules without affecting the size and PDI (Table 3b).

TABLE 3b
Physicochemical properties of glulisine-loaded protamine
nanocapsules post-coated with polysialic acid

| Formulation | | Size (nm) | PDI | ζ-pot (mV) | AE (%) |
|---|---|---|---|---|---|
| NC without cholate | A plus | 315 ± 6 | 0.2 | −28 ± 3 | 54 ± 5 |
| NC with sodium | B plus | 242 ± 4 | 0.2 | −21 ± 2 | 29 ± 3 |
| cholate | | | | | |
| NC with sodium | C plus | 253 ± 7 | 0.2 | −28 ± 2 | 51 ± 6 |
| glycol-cholate | | | | | |

Constant parameters, concentration in the final formulation (mg/ml): Miglyol ® = 11.8; PEGst 40 = 2.4; Protamine = 1, Polysialic acid = 0.17 (pH of final formulations 5-6)

Human insulin was also encapsulated in protamine nanocapsules with an oleic acid oil core. The encapsulation vales were dependent on the type of bile salt (Table 3c).

TABLE 3c
Physico-chemical properties of human insulin-loaded
protamine nanocapsules with an oleic acid core

| | pH of | | | | |
|---|---|---|---|---|---|
| Formulation | Aqueous phase | Size (nm) | PDI | Z-pot (mV) | AE % |
| NC with Na | 10.4 | 251 ± 15 | 0.2 | −19 ± 2 | 14 ± 5 |
| glycocholate | | | | | |
| NC with Na | 10.8 | 181 ± 5 | 0.2 | −27 ± 4 | 37 ± 8 |
| deoxycholate | | | | | |

Constant parameters, concentration in the final formulation (mg/ml): Oleic acid = 11.8; PEGst 40 = 2.4; Protamine = 1, (pH of final formulations 6-7).

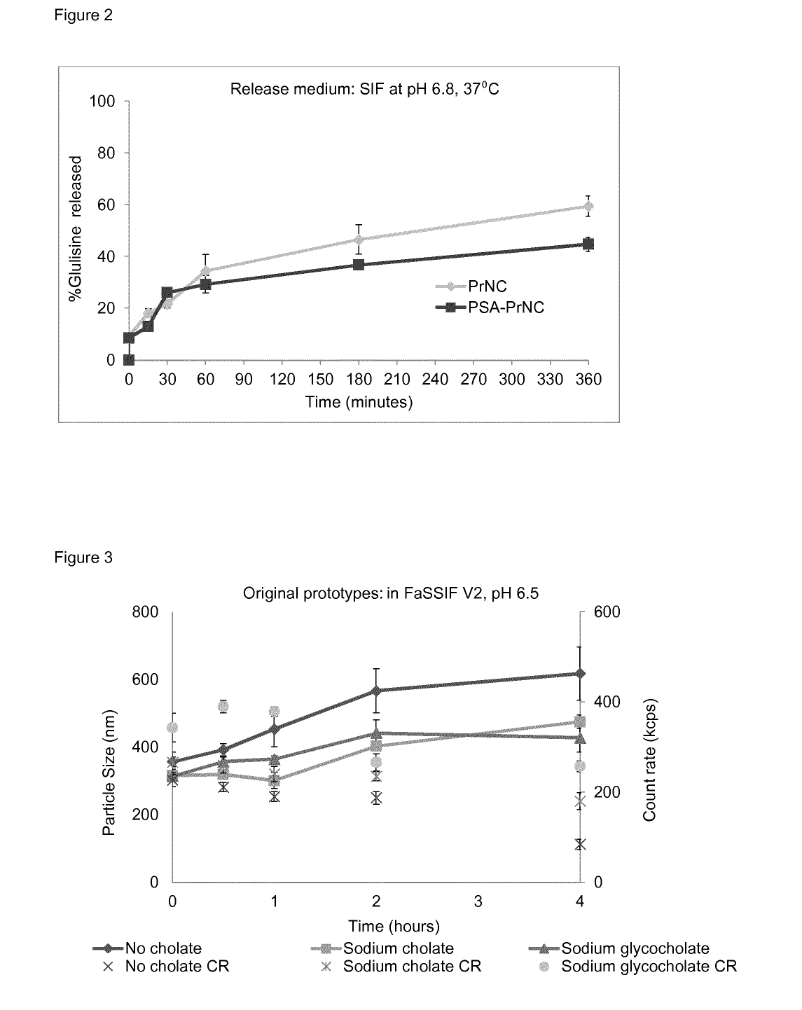

In vitro release studies of insulin glulisine-loaded protamine nanocapsules and polysialic acid-coated protamine nanocapsules were carried out by incubating the nanosystem in SIF. The in vitro release profile in FIG. 2 shows that only 10% of the encapsulated insulin was released at 0 hours. This % insulin released increases gradually up to 60% after 6 hours. This gradual release profile of insulin suggests that protamine nanocapsules release the encapsulated insulin in a controlled manner. The insulin glulisine-loaded nanocapsules were stable in SIF in the presence or absence of pancreatic enzymes.

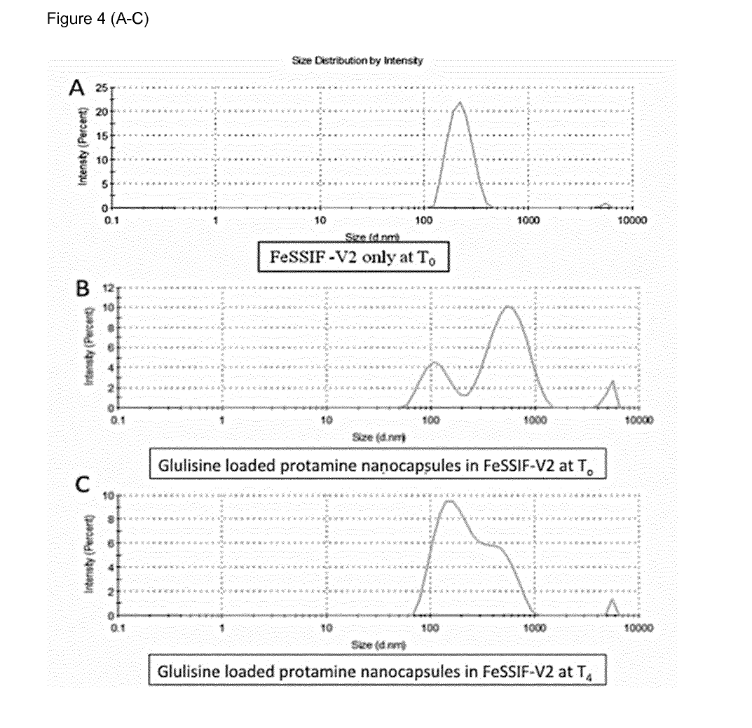

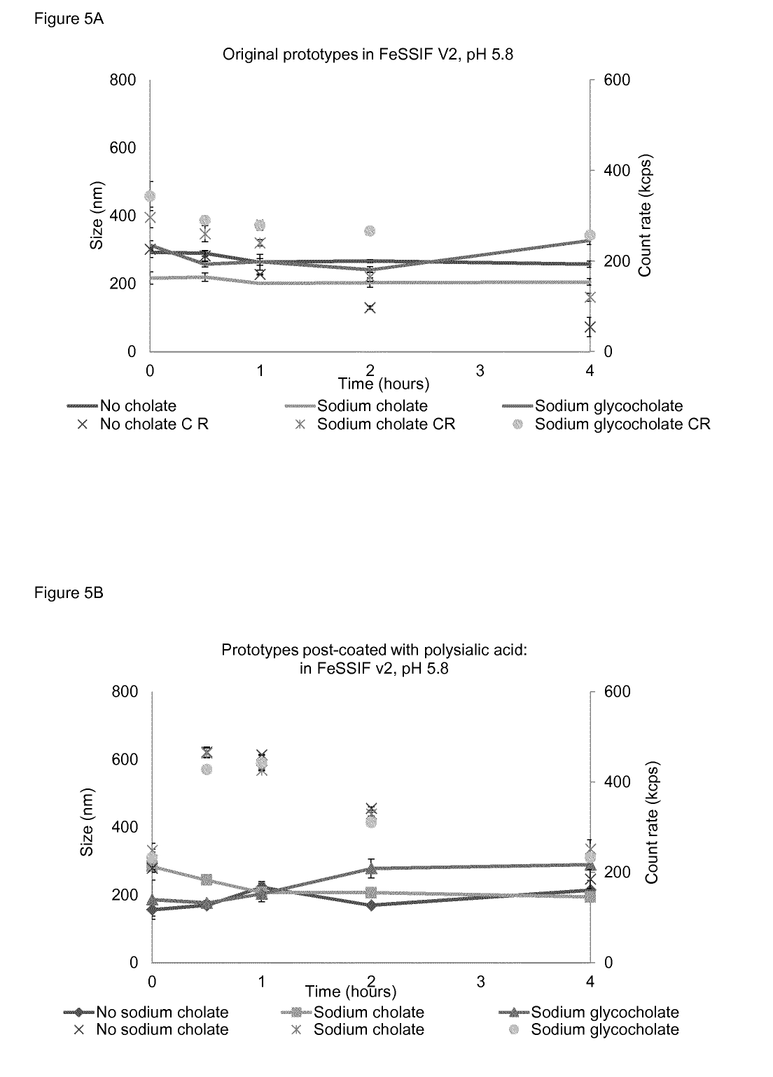

The stability of insulin glulisine-loaded protamine nanocapsules was further determined in more complex media, FaSSIF and FeSSIF, in order to study the potential action of the bile salts and lecithin. Moreover, in the fed state simulated intestinal fluid medium (FeSSIF-V2) a specific amount of pancreatin was added based on a previous literature report [12]. Upon incubation in FaSSIF and FeSSIF media, changes in size and count rate of the protamine nanocapsules were observed. In FaSSIF (FIG. 3) a minor size increase was observed for most formulations, the increase being higher for prototype free of bile salts. On the other hand, in the modified fed state (FeSSIF-V2) the medium itself (FIG. 4A) showed a size similar to that of protamine nanocapsules. This made it difficult to distinguish the peaks corresponding to protamine nanocapsules from those of the medium (FIG. 4B and 4C). In such a situation, an alternative way to measure the stability of the nanocapsules could be to study the stability of the encapsulated insulin. As shown in FIG. 5, the prototype containing glycocholate indicated a rather stable count rate. The prototype without any bile salt showed a reduction in count rate, which may imply that this prototype aggregates in this medium. However, the extra coating layer of polysialic acid promotes the stability of the protamine nanocapsules in the presence of pancreatic enzymes since the count rate was maintained throughout the study (FIG. 5B).

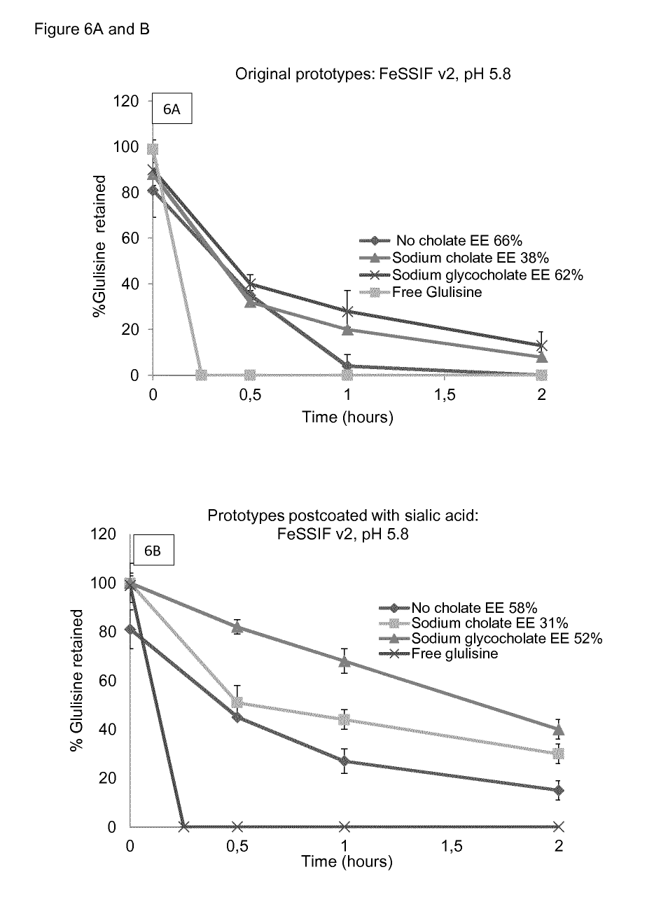

As shown in FIG. 6A, the prototypes containing the bile salts, sodium cholate (prototype B) and sodium glycocholate (prototype C), significantly protect the insulin from proteolysis when compared to prototype A. Evidently, the incorporation of glycocholate in the nanocapsules inhibits protease activity and significantly contributes to the reduction of insulin degradation. Moreover, the presence of polysialic acid outer coating, also improves the ability of the nanocapsules to retain glulisine in the presence of enzymes i.e. minimizes proteolysis (FIG. 6B). The prototype containing sodium glycocholate (C plus, Table 3b) is more stable in complex media due to its enzyme inhibition effects.

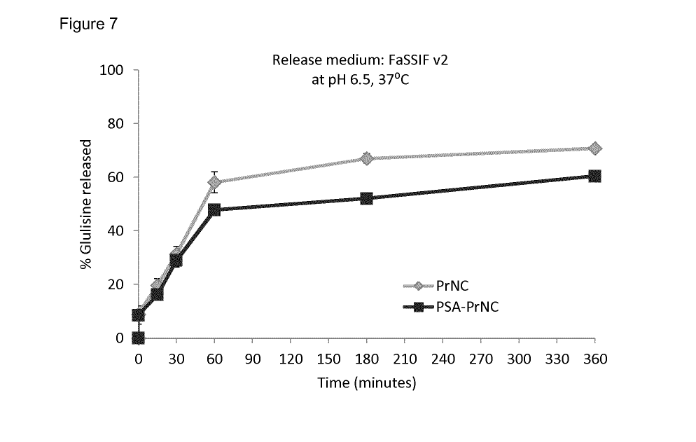

Furthermore, an additional in vitro release study was performed for insulin glulisine-loaded protamine nanocapsules and polysialic acid coated protamine nanocapsules using a more complex medium (FaSSIF v2). As noted in FIG. 2, the results that the loaded insulin is released in a controlled manner from the nanocapsules in the simulated medium (FIG. 7).

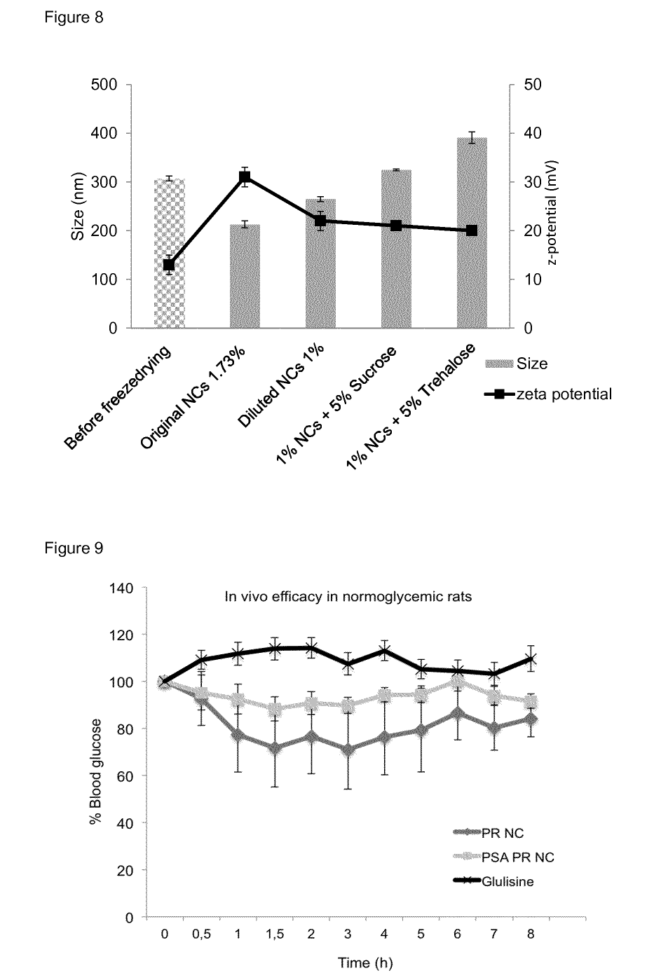

The resuspended dispersions of blank and insulin glulisine-loaded protamine nanocapsules showed physicochemical properties, size and zeta potential, that were similar to those of the pre-freeze dried systems (FIG. 8).

We have also performed a study to analyze the influence of the amount of the constituents of the nanocapsules on their physicochemical and loading properties. The results presented in Table 4a indicate that, in general, the properties the nanocapsules remain unchanged, however, the incorporation of sodium glycocholate and PEGst-40 in high amounts compromises the association efficiency of glulisine (Table 4a)

TABLE 4A
Effects of each component on the physicochemical properties of protamine
nanocapsules on particle size, charge and peptide loading. Formulation
codes A-F denominate different SGC-PEGst-protamine ratios

| | Composition (mg) | | | | | | | | Final |
|---|---|---|---|---|---|---|---|---|---|
| | | PEG- | | Size | | ζ-pot | Final | | loading |
| Code | SGC | st | Prot. | (nm) | PDI | (mV) | pH | EE % | (%) |
| A | 5 | 14 | 10 | 443 ± 186 | 0.3 | −1 ± 2 | 5.04 | 69 ± 5 | 1.2 |
| B | | 14 | 15 | 339 ± 42 | 0.3 | +1 ± 6 | 5.95 | 71 ± 14 | 1.1 |
| C | | 16 | 15 | 394 ± 39 | 0.3 | +0.5 ± 3 | 5.72 | 63 ± 8 | 1.0 |
| D | 7 | 14 | 10 | 245 ± 18 | 0.1 | −1 ± 1 | 5.59 | 25 ± 15* | 0.4 |
| E | | 16 | 10 | 383 ± 60 | 0.3 | +1 ± 0.5 | 5.51 | 64 ± 10 | 1.0 |
| F | | 16 | 15 | 317 ± 50 | 0.2 | −2 ± 1 | 5.57 | 62 ± 6 | 0.96 |

Constant parameters, concentration in the final formulation (mg/ml): Miglyol = 11.8; Initial glulisine loading 1.2%. n = 3 or above for each prototype.
*Precipitated

We have also explored the effect of the pH of the external aqueous phase on the properties of the nanocapsules. In contrast to control nanocapsules (i.e. containing no peptide), it was observed that when the pH of the aqueous phase was around the PI (pH 4.75-5.5) of glulisine, the encapsulation of the peptide was increased and this increase was associated to an increase in the particle size (Table 4B). Overall, when the pH of the external phase was between 5.5 and 6.6, the nanocapsules have an acceptable size and EE %.

TABLE 4B
Influence of pH of the aqueous phase on
physicochemical properties of prototype B

| pH of | | | | | |
|---|---|---|---|---|---|
| aqueous phase | Size (nm) | PDI | ζ-pot (mV) | Final pH | EE % |
| 4.72 | 877 ± 65 | 0.3 | −1± | 5.11 | 74 ± 9 |
| 5.54 | 605 ± 70 | 0.3 | −2± | 5.39 | 70 ± 11 |
| 6.58 | 343 ± 13 | 0.2 | −1± | 5.61 | 68 ± 6 |
| 7.56 | 231 ± 8 | 0.1 | −1± | 6.53 | 18 ± 8* |

Constant parameters, concentration in the final formulation (mg/ml): Sodium glycocholate = 1 mg/ml Miglyol = 11.8; PEGst 40 = 3.2 mg/ml; Protamine = 3 mg/ml, Initial glulisine loading 1.2%.
*Precipitated.

Prototype B (in Table 4A), possessing acceptable size and EE % for glulisine, was used to carry out further studies on protamine nanocapsules after confirming the stability in SIF supplemented with 1% pancreatin. These nanocapsules were stable in SIF/pancreatin as the size and count rate remained constant for 2 hours. Stability study of these prototypes during storage at 4° C. is ongoing.

Protamine and polysialica acid-cloated protamine nanocapsules were labelled with a fluorescent marker in order to investigate their mechanistic behavior both, in vitro and in vivo. For this purpose protamine nanocapsules were labeled with 5-TAMRA, SE (5-Carboxytetramethylrhodamine, succinimidyl ester, single isomer, Mw=527.5, λabs=548 nm, λem/exc=570/554 nm). Protamine sulphate was covalently linked with 5-TAMRA. This conjugate was purified by dialysis for 72 hours to remove free TAMRA and freeze-dried. Conjugation between protamine and 5-TAMRA was confirmed using Ultraviolet-Visible spectroscopy (UV) and Fourier Transform Infrared spectroscopy (FTIR). The disappearance of the protamine amide peak at 1052 cm-1 in the 5-TAMRA-protamine and the appearance of the carboxamide peak at 1277 cm-1 confirmed conjugation of 5-TAMRA and protamine. Finally, protamine nanocapsules were prepared according to the standard method (Table 5).

TABLE 5
Physicochemical characteristics of TAMRA-protamine and
TAMRA-polysialic acid-protamine nanocapsules (NC)

| | | | | | Stability |
|---|---|---|---|---|---|
| Sample name | Size (nm) | PDI | Z-pot (mv) | EE % | (weeks)* |
| Blank Tamra- | 199 ± 15 | 0.1 | +5.7 | N/A | 2 |
| PrNC | | | | | |
| Blank PSA- | 211 ± 19 | 0.1 | −1 | N/A | 2 |
| Tamra-PrNC | | | | | |
| glulisine- | 315 ± 73 | 0.3 | +16 ± 5 | 56 ± 5 | 2 |
| Tamra-PrNC | | | | | |
| glulisine-PSA- | 307 ± 93 | 0.1 | −1 ± 3 | 42 ± 10 | 2 |
| Tamra-PrNC | | | | | |

*ongoing study. n = 3 or more

In order to determine the in vivo efficacy of glulisine-loaded protamine and polysialic acid-coated protamine nanocapsules, these formulations were administered in vivo to normoglycemic male Sprague-Dawley rats via an intraduodenal canule (250 g, 6 hours fasted, n=5-12). A single dose of 501U/kg glulisine, encapsulated or in solution, was administered to each animal in a maximum volume of 300 p1 by slow injection through a needle inserted in the intraduodenal cathether. Blood glucose levels were monitored for 8 hours using a glucometer (One Touch Ultra, Johnson and Johnson) and using a colorimetric method (SpinReact). Different groups of animals receiving the same formulation were treated in different days, what might explain the large error observed in the graphs presented in FIG. 9 (pool data of 1- to 3 individual experiments). The results indicate that, compared to the same dose of intraduodenally administered glulisine solution, both formulations induced significant reduction of blood glucose levels for several hours.

### Example 2

Example 2 relates to the influence of the composition of Polyarginine nanocapsules on their physicochemical properties and insulin loading capacity. Insulin encapsulation capacity was evaluated using Zn-hexameric insulin (human insulin) provided by Sanofi-Aventis Deutschland GmbH.

### Materials and Methods

Polyarginine (PArg) nanocapsules were prepared by a modified solvent displacement technique, as previously described (M. V. Lozano, G. Lollo, M. Alonso-Nocelo, J. Brea, A. Vidal, D. Torres & M. J. Alonso. Polyarginine nanocapsules: a new platform for intracellular drug delivery. Journal of Nanoparticle Research, 2013, 15(3) 1515). The lipid phase consisted of oil (oleic acid, Sigma-Aldrich or Croda) and surfactants (Sorbitan Oleate (Span® 80, Sigma-Aldrich or Croda) and Sodium deoxycholate, (Sigma-Aldrich or New Zealand Pharmaceuticals). The aqueous phase consisted of polyarginine (PArg) of different molecular weight (one chloride salt of 26-37kDa, PTS, Spain and another chloride salt of 5-15kDa, Sigma-Aldrich) in combination with Poloxamer 188 (Sigma-Aldrich or BASF).

For the encapsulation of insulin, human insulin was incorporated into the organic phase in a small volume of 0.01 N HCl. The organic phase was then transferred immediately to an aqueous phase containing polyarginine as coating polymer, leading to the spontaneous formation of the nanocapsules. The organic solvents were removed by rotaevaporation. The variable parameter investigated was the pH of the aqueous phase.

The average diameter and polydispersity index (PDI) of polyarginine nanocapsules were measured by dynamic light scattering on a zeta sizer Nano series DTS 1060 (Malvern instruments), after dilution with ultrapure water. The zeta potential was measured by laser-Doppler anemometry after diluting the samples with KCl 1 mM (Zetasizer®, NanoZS, Malvern Instruments, Malvern, UK).

The stability of insulin-loaded polyarginine nanocapsules upon incubation at 37° C. in different media was studied. The media were: Simulated intestinal fluid (SIF) pH 6.8 in the presence and absence of pancreatin. The size distribution, polydispersity index (PDI) and count rate of the nanocapsules were monitored by dynamic light scattering on a zeta sizer Nano series DTS 1060 (Malvern instruments).

The colloidal stability of the blank nanocapsules was followed during a period of 2 months at different temperatures (4° C., 20° C. and 37° C.). Samples were withdrawn at predetermined time intervals, and the particle size and zeta potential were determined as described above.

The insulin association efficiency (%) was determined using the direct method, which involves the extraction of insulin from the nanocapsules. Insulin loaded nanocapsules (0.1 ml) were digested using a combination of acetonitrile (0.16 ml), 0.1% TFA (0.64 ml) and Triton-X100 (0.1 ml). Then the formulation was vortexed at a high speed to obtain a clear aliquot. The concentration of insulin in this aliquot was determined by reverse phase HPLC. The insulin entrapment or association efficiency was calculated according to equation 1. The insulin final loading or loading capacity was calculated according to equation 2.

$$\begin{matrix}Entrapment efficiency ( EE ) [ \% ] = \frac{Amount of insulin in destructed nanocapsules}{Amount of insulin added} 100 & Equation 1 \\ Loading capacity [ \% ] = \frac{Theoretical mass of insulin EE}{Actual mass of nanocapsules} 100 & Equation 2\end{matrix}$$

The in vitro release of human insulin from the nanocapsules was determined upon incubation in simulated intestinal fluid (SIF, pH 6.8) at 37° C. At specified time intervals (0, 1, 3 and 6 hours), the supernatant was collected by centrifugation. The concentrations of human insulin in the supernatant were determined by reverse phase HPLC method, and the total amount of insulin released from the nanocapsules was calculated.

Blank and human insulin loaded PArg nanocapsules (1% w/v) were lyophilized (Labconco Corp, USA) in presence of sucrose at 5% (w/v). The freeze-dried formulations were resuspended with ultrapure water by manual shaking and their physicochemical characteristics were evaluated.

In some formulations, pH 5.5 buffers were used to formulate the nanocapsules. The buffers were prepared by titration of acetic acid or citric acid with sodium hydroxide at 25° C.

### Results

The properties of blank nanocapsules are shown in Table 6. The nanocapsules exhibited a small size and a positive zeta potential and these properties were not affected by the PArg molecular weight. These blank nanocarriers were stable upon incubation in SIF with or without pancreatin.

TABLE 6
Physicochemical properties of blank PArg nanocapsules
(NCs) using PArg of two different molecular weights

| | PArg | | | | Stability | Stability |
|---|---|---|---|---|---|---|
| | MW | Size | | Z-Pot | SIF pH | SIF + |
| Properties | (kDa) | (nm) | PdI | (mV) | 6.8 | pancreatin |
| Blank NCs | 5-15 | 178 ± 3 | <0.2 | +11 ± 2 | Stable in | Stable for |
| without | 26-37 | 195 ± 1 | <0.2 | +10 ± 1 | 6 h | 6 h |
| deoxycholate | | | | | | |

Constant parameters, concentration in the final formulation (mg/ml): Oleic Acid = 12.2; Span ®80 = 4; Pluronic ®F68 = 5; PArg = 1

Table 7 shows the properties of insulin loaded PArg nanocapsules. The results show that the pH of aqueous medium influenced significantly the encapsulation efficiency. The co-encapsulation of sodium deoxycholate in the organic phase also made a difference in the loading efficiency. These results suggest the ionic interaction of the external aqueous phase and inner oily core may have to do with the loading efficiency. Also, the deprotonation of insulin at the final formulation pH may increase its affinity to the oil, which leads to a better drug encapsulation.

TABLE 7
Physicochemical properties and loading capacity of human
insulin-loaded PArg nanocapsules (NC): variables, physicochemical
characteristics and loading capacity of insulin

| | | | | | | | | Final |
|---|---|---|---|---|---|---|---|---|
| | | pH | pH NC | Size | | Z-pot | | loading |
| Properties | | (aq) | (Final) | (nm) | PdI | (mV) | AE (%) | (%) |
| NCs without | A | 4.9 | 3-3.3 | 213 ± 5 | <0.2 | +12 ± 1 | <10 | — |
| deoxy- | B | 10.8 | 3.3-3.6 | 242 ± 5 | <0.2 | −19 ± 1 | 31 ± 15 | 1.4 ± 0.7 |
| cholate | C | 11.2 | 3.6-4 | 217 ± 17 | <0.2 | −40 ± 3 | 61 ± 14 | 2.8 ± 0.6 |
| NCs with | D | 4.9 | 3-3.5 | 213 ± 27 | <0.2 | +7 ± 4 | <10 | — |
| deoxy- | E | 10.8 | 5-6 | 178 ± 20 | <0.2 | −30 ± 2 | 81 ± 6 | 3.7 ± 0.3 |
| cholate | F | 11.2 | 6.5-8 | 155 ± 12 | <0.2 | −51 ± 8 | 40 ± 23 | 1.8 ± 1.0 |

Contant parameters, concentration in the final formulation (mg/ml): Oleic Acid = 11.2; Span ®80 = 4; Pluronic ®F68 = 5; PArg = 1, sodium deoxycholate = 0.5

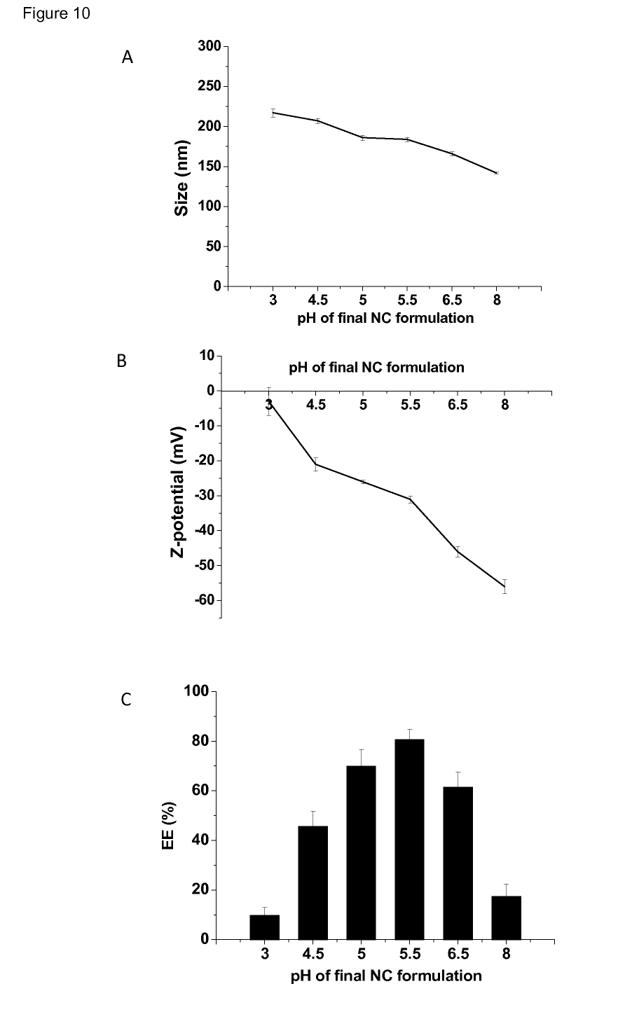

In a different study the influence of the pH of the external aqueous phase in the physicochemical properties of the nanocapsules was investigated. For this, the formulation of PArg nanocapsules containing deoxycholate was chosen. The results in FIGS. 10 A, B and C illustrate the influence of the final formulation pH on the particle size, the Z-potential and the EE % respectively. The conclusion was that a pH 5.5, close to the isoelectric point of insulin, was the optimum pH that led to appropriate encapsulation efficiency.

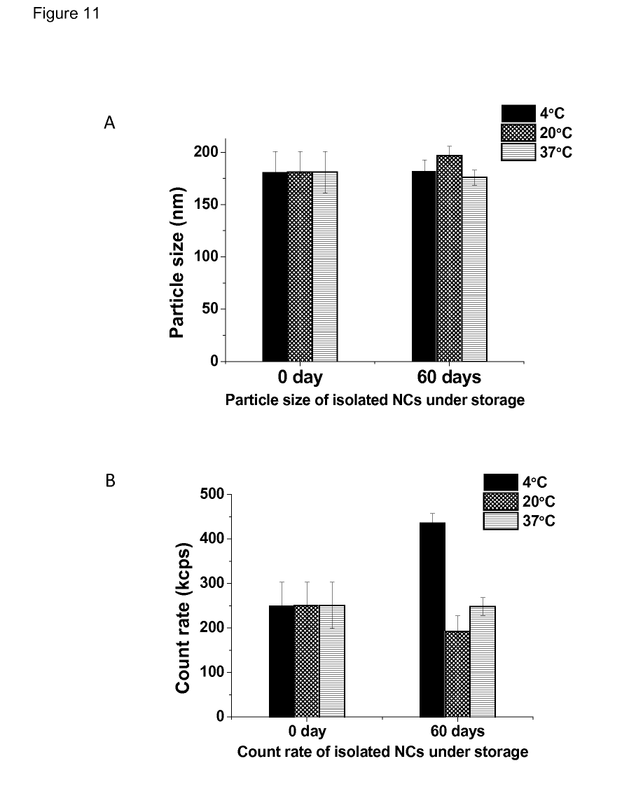

The results showed that PArg NCs were stable at 4° C., 20° C. and 37° C. for at least 2 months showing no significant changes in their physicochemical characteristics (FIG. 11). These nanocapsules could also be freeze-dried with 5% sucrose well resuspended in water, maintaining the physicochemical characteristics.

In order to control the pH of the nanoparticles suspending medium a number of buffers at different concentrations were explored. Acetate buffer was found to be appropriate in terms of preserving the physico-chemical properties and insulin loading of PArg nanocapsules. As shown in Table 8, a concentration 20 mM acetate buffer was found to be adequate for the preservation of the nanocapsules properties. The encapsulation efficiency for this formulation was 88±5%.

TABLE 8
Physico-chemical properties of PArg NCs prepared with pH 5.5 acetate buffer at different buffer concentrations.
D (10), D (50) and D (90) means that 10%, 50% and 90% of the NCs are below this size, n = 10 for
non-buffer (prototype E in Table 7) and 20 mM buffer based nanocapsule; n = 3 for the rest.

| Buffer | Ionic | Size | | Z-pot | Count rate | | | | Final |
|---|---|---|---|---|---|---|---|---|---|
| concentration | force | (nm) | PdI | (mV) | (kcps) | D(10) | D(50) | D(90) | pH |
| Non buffer | — | 178 ± 20 | 0.12 | −31 ± 3 | 318 ± 108 | 126 ± 7 | 192 ± 17 | 300 ± 51 | 5.0-6.0 |
| 10 mM | 0.004M | Aggregation during preparation | | | | | | | |
| 20 mM | 0.017M | 185 ± 6 | 0.15 | −24 ± 3 | 411 ± 76 | 115 ± 4 | 200 ± 4 | 347 ± 17 | 5.2-5.4 |
| 30 mM | 0.025M | 187 ± 12 | 0.18 | −21 ± 1 | 228 ± 45 | 111 ± 5 | 201 ± 18 | 395 ± 99 | 5.2-5.4 |
| 50 mM | 0.043M | 197 ± 6 | 0.17 | −20 ± 1 | 282 ± 58 | 121 ± 9 | 216 ± 8 | 387 ± 16 | 5.4-5.5 |

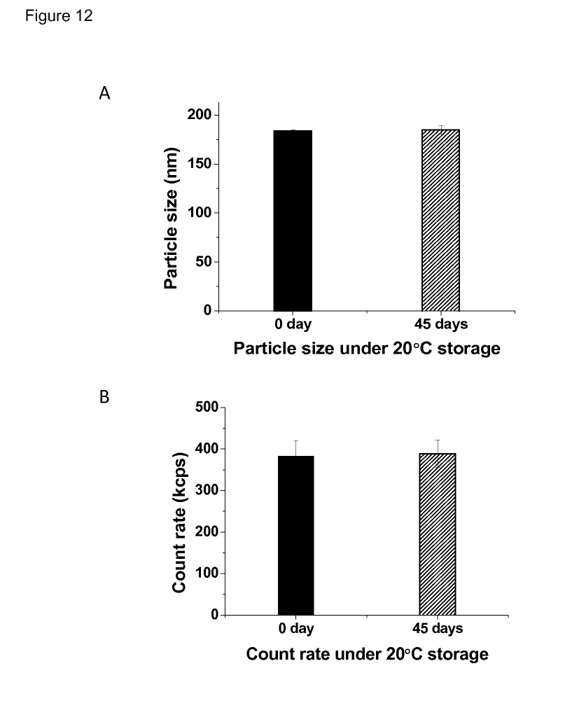

The insulin-loaded PArg NCs prepared with 20 mM acetate buffer were found to be stable at 20° C. for at least 45 days (FIG. 12), while at 4° C. there is a tendency of aggregation after 1 week storage. Count rate in the figures indicates the concentration of the NCs.

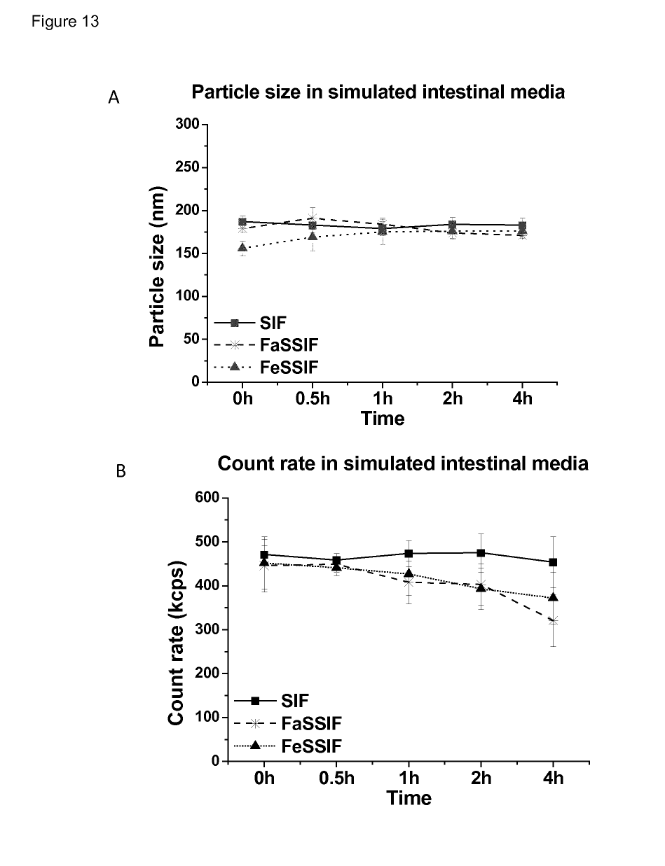

An additional requirement for PArg nanocapsules to be used for the oral administration of insulin was their colloidal stability. As shown in FIG. 13, PArg NCs have good colloidal stability in Simulated Intestinal Fluid (SIF, pH 6.8, European Pharmacopeia, 2011), Fasted State Simulated Intestinal Fluid (FaSSIF-V2, pH 6.5) and Fed State Simulated Intestinal Fluid (FeSSIF-V2, pH 5.8, with enzyme). Nanocapsules prepared with and without 20 mM acetate buffer showed the same colloidal stability profile in these intestinal media.

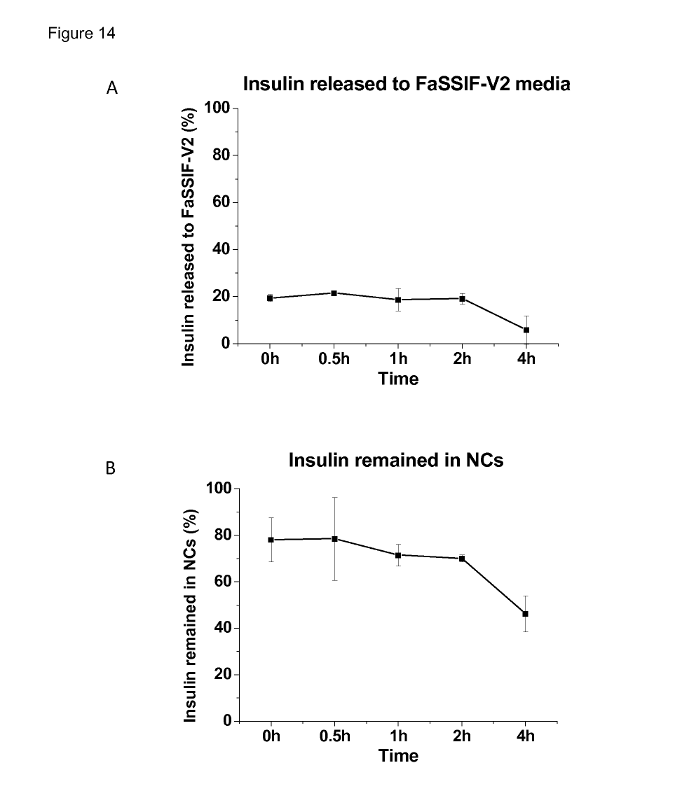

The in vitro release studies were preformed for PArg nanocapsules prepared with 20 mM acetate buffer FaSSIF-V2 medium. The results in FIG. 14, indicate that a 22% initial insulin release, followed by a gradual release up to 54% until 4 h. In FIG. 13, it is also shown that amount of insulin that remains retained in the nanocapsules over the incubation time. By comparing the % of insulin released and that of insulin retained in the nanocapsules, it could be speculated that there is a partial degradation/aggregation of insulin once released to FaSSIF-V2 from the nanocapsules. Irrespective of this, the conclusion is that PArg nanocapsules provide a sustained release of the encapsulated insulin.

1. A nanocapsule system that comprises:
a. a surface layer that comprises a cationic charged polymer; and
b. optionally a second surface layer that comprises a negatively charged polymer; and
c. a lipid core that comprises at least one lipophilic compound; and
d. at least one surfactant or a mixture of surfactants; and
e. at least one active pharmaceutical ingredient selected from the group consisting of insulin, insulin analogue, insulin derivative, glucagon-like peptide-1 receptor agonist (GLP1R agonist), dual GLP-1 receptor/glucagon receptor agonist, and any combination thereof.

2. The nanocapsule system as claimed in claim 1, wherein the cationic charged polymer is selected from the group consisting of peptides, polyaminoacids, polylysine, polyarginine, lixisenatide, protamine and combinations thereof.

3. The nanocapsule system as claimed in claim 1, wherein the negatively charged polymer is selected from the group consisting of polysialic acid, polyacrylic acid, hyaluronic acid, polyglutamic acid, alginic acid, polyglucuronic acid, and xanthan gum.

4. The nanocapsule system as claimed in any of claims 1 to 3, wherein the surfactant or the mixture of surfactants is characterized by possessing a hydrophilic-lipophilic balance (HLB) above 8.

5. The nanocapsule system as claimed in any of claims 1 to 4, wherein the at least one surfactant or the mixture of surfactants comprises at least one surfactant which is selected from the group consisting of sorbitan esterified with at least one fatty acids and ethoxylates thereof, fatty acid esters, fatty acid salts, gum tragacanth, bile salts and bile salt derivatives and poloxamers.

6. The nanocapsule system as claimed in claim 5, wherein
a. at least one surfactant is a sorbitan esterified with at least fatty acid or ethoxylates thereof selected from the group consisting of sorbitan monooleate, sorbitan monolaurate, sorbitan monostearate, sorbitan trioleate, sorbitan sesquiolate, sorbitan monopalmitate, sorbitan isostearate, polyoxyethylene sorbitan monooleate (polysorbate 80), polyoxyethylene sorbitan monolaurate (polysorbate 20), polyoxyethylene sorbitan monostearate, polyoxyethylene sorbitan monooleate, polyoxyethylene sorbitan tristearate, polyoxyethylene sorbitan trioleate, polyoxyethylene sorbitan monolaurate, polyoxyethylene sorbitan monopalmitate and any combination thereof;
or
b. at least one surfactant is a fatty acid ester or a fatty acid salt selected from the group consisting of polyethylene glycol monostearate, polyethylene glycol stearate 40, polyethylene glycol dilaurate, polyethylene glycol monopalmitate, polyethylene glycol stearate 100, polyethylene glycol-15-hydroxystearate, D-alpha-tocopheryl polyethylene glycol succinate (TPGS), triethanolammonium oleate, sodium oleate, sodium lauryl sulphate, triethanolamine oleate, and sodium dodecyl sulfate, lithium dodecyl sulfate, sodium oleate, and any combination thereof;
or
c. at least one surfactant is a poloxamer selected from the group consisting of Poloxamer 124, Poloxamer 188, Poloxamer 237, Poloxamer 238 (HLB 28), Poloxamer 278 (HLB 28), Poloxamer 338, Poloxamer 407, and any combination thereof;
or
d. at least one surfactant is a bile salt or bile salt derivative selected from the group consisting of sodium cholate, sodium deoxycholate, sodium glyocholate, sodium taurocholate, and sodium taurodeoxycholate.

7. The nanocapsule system as claimed in any of claims 1 to 6, wherein the surfactant or the mixture of surfactants comprises at least one surfactant selected from the group consisting of sodium cholate, sodium glycocholate, sodium deoxycholate, polyethylene glycol stearate, polyethylene glycol-15-hydroxystearate, D-alpha-tocopheryl polyethylene glycol succinate (TPGS), polyoxyethylene sorbitan monooleate, polyoxyethylene sorbitan monolaurate, poloxamer, and any combination thereof.

8. The nanocapsule system as claimed in any of claims 1 to 7, wherein the lipophilic compound is selected from the group consisting of peanut oil, cottonseed oil, olive oil, castor oil, soybean oil, safflower oil, geranium oil, palm oil, alpha-tocopherol (vitamin E), oleic acid, linoleic acid, isopropyl myristate, squalene, caprylic/capric triglyceride, linoleoyl macrogol-6 glycerides (corn oil PEG-6 esters), triglycerides medium chain, glyceryl oleate, glyceryl linoleate, glycerol monooleate, and any combination thereof.

9. The nanocapsule system as claimed in any of claims 1 to 8, wherein
a. the insulin is human insulin; and/or
b. the insulin analogue is selected from the group consisting of insulin aspart, insulin lispro, insulin glargine and insulin glulisine or any combinations thereof; and/or
c. the insulin derivative is selected from the group consisting of insulin detemir and insulin degludec; and/or
d. the glucagon-like peptide-1 receptor agonist (GLP1 R agonist) is selected from the group consisting of exendin-4, liraglutide, lixisenatide, dulaglutide, albiglutide, semaglutide, and taspoglutide and any combinations thereof.

10. A pharmaceutical composition that comprises the nanocapsule system as claimed in any of claims 1 to 9 and optionally one or more further active pharmaceutical ingredients and optionally one or more pharmaceutically acceptable excipients.

11. The nanocapsule system as claimed in any one of claims 1 to 9 and/or the pharmaceutical composition as claimed in claim 10, characterized in that it is lyophilized.

12. A kit comprising one or more separate packages of
a. the nanocapsule system as claimed in any one of claims 1 to 9 or the pharmaceutical composition as claimed in claim 10; and
b. at least one further active pharmaceutical ingredient; and optionally
c. a medical device.

13. The nanocapsule system as claimed in any one of claims 1 to 9 or the pharmaceutical composition as claimed in claim 10 or the kit as claimed in claim 12
a. for use in the treatment of diabetes mellitus; and/or
b. for use in the treatment of hyperglycemia; and/or
c. for use in lowering blood glucose level.

14. The nanocapsule system as claimed in any one of claims 1 to 9 or the pharmaceutical composition as claimed in claim 10 or the kit as claimed in claim 12 for subcutaneous, transdermal, buccal, oral, pulmonary or nasal administration.

15. A method of treating diabetes mellitus or a method of treating hyperglycemia or a method of lowering blood glucose levels in a subject in need thereof comprising administering the nanocapsule system as claimed in any one of claims 1 to 9 or the pharmaceutical composition as claimed in claim 10.

16. A medical device for administering the nanocapsule system as claimed in any one of claims 1 to 9 or the pharmaceutical composition as claimed in claim 10.

17. A method for producing the nanocapsule system as claimed in any one of claims 1 to 9 comprising the emulsification of an internal lipophilic phase containing the lipophilic compound and an external water phase containing the cationic charged polymer, optionally a negatively charged polymer, and the surfactant or the mixture of surfactants, and the at least one active pharmaceutical ingredient.

18. The method as claimed in claim 17, wherein the at least one active pharmaceutical ingredient is dispersed in the lipophilic phase either as a powder or as an aqueous solution.

19. The method as claimed in claim 17, wherein the at least one active pharmaceutical ingredient is dispersed either in the lipophilic phase either as a powder or as an aqueous solution.
